(19) United States
(12) Patent Application Publication
(10) Pub. No.: US 2025/0376718 A1
(43) Pub. Date: Dec. 11, 2025

(54) HIGH RESOLUTION MULTIPLEX METHOD FOR DETECTING AT LEAST TWO TARGETS WITH A DISTANCE OF BEYOND THE DIFFRACTION LIMIT IN A SAMPLE

(71) Applicant: Resolve BioSciences GmbH, Monheim am Rhein (DE)

(72) Inventors: Cheng Frank Zhong, Monheim am Rhein (DE); Christian KORFHAGE, Monheim am Rhein (DE); Frank REINECKE, Monheim am Rhein (DE); Stephan Tirier, Monheim am Rhein (DE)

(73) Assignee: Resolve BioSciences GmbH, Monheim am Rhein (DE)

(21) Appl. No.: 18/878,275

(22) PCT Filed: Jun. 29, 2023
(86) PCT No.: PCT/EP2023/067782

§ 371 Date: Dec. 23, 2024

Related U.S. Application Data
(60) Provisional application No. 63/357,176, filed on Jun. 30, 2022.
(60) Provisional application No. 63/487,309, filed on Feb. 28, 2023.

(51) Int. Cl. C12Q 1/6841; C12Q 1/6818; C12Q 1/6886; G01N 21/64

(52) U.S. Cl. CPC C12Q 1/6841; C12Q 1/6818; C12Q 1/6886; G01N 21/6428; G01N 21/6458; C12Q 2600/156; C12Q 2600/16; G01N 2021/6439

(57) ABSTRACT

The technology provided herein relates to high resolution multiplex methods and kits for detecting different analytes in a sample, such as by sequential signal-encoding of said analytes. The methods allows a differentiation of targets which distance is below the diffraction limit of optical microscopes, that is, targets with spatial optical overlap. The disclosed methods also include in vitro methods for screening, identifying and/or testing a substance and/or drug and in vitro methods for diagnosis of a disease, and an optical multiplexing system.

### RELATED APPLICATIONS

The present application claims the benefit of priority to U.S. Prov Ser. No. 63/357,176, filed Jun. 30, 2022, the contents of which are hereby incorporated by reference in their entirety, and the present application claims the benefit of priority to U.S. Prov Ser. No. 63/487,309, filed Feb. 28, 2023, the contents of which are hereby incorporated by reference in their entirety.

### FIELD OF THE DISCLOSURE

The technology provided herein relates to high resolution multiplex methods and kits for detecting different analytes in a sample, such as by sequential signal-encoding of said analytes or other approach. The technology allows a differentiation of targets even if separated by a distance that is below the diffraction limit of optical microscopes or of the wavelengths used to detect the targets, that is, targets with spatial optical overlap. The disclosed methods also include in vitro methods for screening, identifying and/or testing a substance and/or drug and in vitro methods for diagnosis of a disease or discovering a novel disease factor, and an optical multiplexing system.

### BACKGROUND

The analysis and detection of small quantities of analytes in biological and non-biological samples has become a routine practice in the clinical and analytical environment. Numerous analytical methods have been established for this purpose. Some of them use encoding techniques assigning a particular readable code to a specific first analyte which differs from a code assigned to a specific second analyte. One of the prior art techniques in this field is the so-called ‘single molecule fluorescence in situ hybridization’ (smFISH) essentially developed to detect mRNA molecules in a sample. In Lubeck et al. (2014), Single-cell in situ RNA profiling by sequential hybridization, Nat. Methods11 (4), p. 360-361, the mRNAs of interest are detected via specific directly labeled probe sets. After one round of hybridization and detection, the set of mRNA specific probes is eluted from the mRNAs and the same set of probes with other (or the same) fluorescent labels is used in the next round of hybridization and imaging to generate gene specific color-code schemes over several rounds. The technology needs several differently tagged probe sets per transcript and needs to denature these probe sets after every detection round.

A further development of this technology does not use directly labeled probe sets. Instead, the oligonucleotides of the probe sets provide nucleic acid sequences that serve as initiator for hybridization chain reactions (HCR), a technology that enables signal amplification; see Shah et al. (2016), In situ transcription profiling of single cells reveals spatial organization of cells in the mouse hippocampus, Neuron 92 (2), p. 342-357.

Another technique referred to as ‘multiplexed error robust fluorescence in situ hybridization’ (merFISH) is described by Chen et al. (2015), RNA imaging. Spatially resolved, highly multiplexed RNA profiling in single cells, Science 348 (6233): aaa6090. There, the mRNAs of interest are detected via specific probe sets that provide additional sequence elements for the subsequent specific hybridization of fluorescently labeled oligonucleotides. Each probe set provides four different sequence elements out of a total of 16 sequence elements. After hybridization of the specific probe sets to the mRNAs of interest, the so-called readout hybridizations are performed. In each readout hybridization one out of the 16 fluorescently labeled oligonucleotides complementary to one of the sequence elements is hybridized. All readout oligonucleotides use the same fluorescent color. After imaging, the fluorescent signals are destroyed via illumination and the next round of readout hybridization takes place without a denaturing step. As a result, a binary code is generated for each mRNA species. A unique signal signature of 4 signals in 16 rounds is created using only a single hybridization round for binding of specific probe sets to the mRNAs of interest, followed by 16 rounds of hybridization of readout oligonucleotides labeled by a single fluorescence color.

A further development of this technology improves the throughput by using two different fluorescent colors, eliminating the signals via disulfide cleavage between the readout-oligonucleotides and the fluorescent label and an alternative hybridization buffer; see Moffitt et al. (2016), High-throughput single-cell gene-expression profiling with multiplexed error-robust fluorescence in situ hybridization, Proc. Natl. Acad. Sci. USA. 113 (39), p. 11046-11051.

Xia et al. (2019) (https://doi.org/10.1073/pnas.1912459116) further increased the gene throughput of MERFISH and achieved 10,000 plex using 23 rounds of hybridization and 3 color channels. To reduce the impact of crowding and diffraction limited spots, this version of MERFISH uses expansion microscopy to increase the voxel space in which individual transcripts can be detected.

A technology referred to as ‘intron seqFISH’ is described in Shah et al. (2018), Dynamics and spatial genomics of the nascent transcriptome by intron seqFISH, Cell 117 (2), p. 363-376. There, the mRNAs of interest are detected via specific probe sets that provide additional sequence elements for the subsequent specific hybridization of fluorescently labeled oligonucleotides. Each probe set provides one out of 12 possible sequence elements (representing the 12 ‘pseudocolors’ used) per color-coding round. Each color-coding round consists of four serial hybridizations. In each of these serial hybridizations, three readout probes, each labeled with a different fluorophore, are hybridized to the corresponding elements of the mRNA-specific probe sets. After imaging, the readout probes are stripped off by a 55% formamide buffer and the next hybridization follows. After 5 color-coding rounds with 4 serial hybridizations each, the color-codes are completed.

A further development of this technology, termed ‘seqFISH+’ (https://doi.org/10.1038/s41586-019-1049-y), used the same principle of pseudocolors, but encodes individual transcripts in one of three color channels separately to eliminate chromatic aberrations. To reduce the impact of crowding and diffraction limited spots, seqFISH+dilutes signals into 4 color coding rounds with 20 serial hybridizations each in combination with subpixel localization of spots. Thereby, seqFISH+achieves 10,000plex smRNA-FISH, but with very high false positive rates (FPR=0.22).

Another technology, CosMX™ SMI, (He et al., 2022) (https://doi.org/10.1038/s41587-022-01483-z) utilizes a limited number (˜5) of up to 130 nt long target probes that contain a target binding domain complementary the transcript of interest and four readout domains that bind fluorescent reporters. Reporters consist of branched DNA structures that contain up to 60 fluorophores, thereby complementing for the low number of target probes. Each target probe contains a unique set of four different sequence elements out of a total of 64 sequence elements (16 sequence elements*4 colors). Individual transcripts are binary encoded using a 64-bit barcode and 4 signals per transcript (one per color) over 16 rounds of hybridization. Like the seqFISH technology, CosMX™ uses subpixel localization of spots enabled by the dilution of signals to reduce the impact of crowding and diffraction limited spots.

EP 0 611 828 discloses the use of a bridging element to recruit a signal generating element to probes that specifically bind to an analyte. A more specific statement describes the detection of nucleic acids via specific probes that recruit a bridging nucleic acid molecule. This bridging nucleic acids eventually recruit signal generating nucleic acids. This document also describes the use of a bridging element with more than one binding site for the signal generating element for signal amplification like branched DNA.

Player et al. (2001), Single-copy gene detection using branched DNA (bDNA) in situ hybridization, J. Histochem. Cytochem. 49 (5), p. 603-611, describe a method where the nucleic acids of interest are detected via specific probe sets providing an additional sequence element. In a second step, a preamplifier oligonucleotide is hybridized to this sequence element. This preamplifier oligonucleotide comprises multiple binding sites for amplifier oligonucleotides that are hybridized in a subsequent step. These amplifier oligonucleotides provide multiple sequence elements for the labeled oligonucleotides. This way a branched oligonucleotide tree is build up that leads to an amplification of the signal.

A further development of this method referred to as is described by Wang et al. (2012), RNAscope: a novel in situ RNA analysis platform for formalin-fixed, paraffin-embedded tissues, J. Mol. Diagn. 14 (1), p.22-29, which uses another design of the mRNA-specific probes. Here two of the mRNA-specific oligonucleotides have to hybridize in close proximity to provide a sequence that can recruit the preamplifier oligonucleotide. This way the specificity of the method is increased by reducing the number of false positive signals.

Choi et al. (2010), Programmable in situ amplification for multiplexed imaging of mRNA expression, Nat. Biotechnol. 28 (11), p. 1208-1212, disclose a method known as ‘HCR-hybridization chain reaction’. The mRNAs of interest are detected via specific probe sets that provide an additional sequence element. The additional sequence element is an initiator sequence to start the hybridization chain reaction. Basically, the hybridization chain reaction is based on metastable oligonucleotide hairpins that self-assemble into polymers after a first hairpin is opened via the initiator sequence.

A further development of the technology uses so called split initiator probes that have to hybridize in close proximity to form the initiator sequence for HCR, similarly to the RNAscope technology, this reduces the number of false positive signals; see Choi et al. (2018), Third-generation in situ hybridization chain reaction: multiplexed, quantitative, sensitive, versatile, robust. Development 145 (12).

Mateo et al. (2019), Visualizing DNA folding and RNA in embryos at single-cell resolution, Nature Vol, 568, p. 49ff., disclose a method called ‘optical reconstruction of chromatin structure (ORCA). This method is intended to make the chromosome line visible.

EP 2 992 115 B1 describes a method of sequential single molecule hybridization and provides technologies for detecting and/or quantifying nucleic acids in cells, tissues, organs or organisms through sequential barcoding.

The methods known in the art, however, have numerous disadvantages. In particular, they are inflexible, expensive, complex, time consuming and quite often provide non-accurate results. In particular, the encoding capacities of the existing methods are low and do not meet the requirements of modern molecular biology and medicine.

Furthermore, the methods known so far do not provide a reliable signal in cases in which the target, for example spatially overlapping transcripts, cannot be differentiated by optical methods due to the diffraction limit, that is, the minimal distance at which two signal spots can be differentiated in a microscope. However, if the detection of spots is limited by the resolution of the microscope, it is not possible to identify transcripts that are very close to each other. This is relevant for detection of fusion genes (cancer) or for detection of co-localization of different transcript types (e.g. transcriptional hubs in the nucleus), among others. In addition, transcripts with higher expression levels are also problematic because the high number of signals affects the detection of other (especially lowly expressed) genes.

Some methods are limited not only by the diffraction limit of the wavelength used for detection, but also the compositions used for detection. Bead-based sequencing approaches, for example, are limited by the size of the bead used in transcript detection, which is often far larger than the detection wavelength. In these technologies, multiple transcripts or other analytes may be localized to a common bead, but may not be detected or localized to a sample, or to one another, beyond the resolution limit of the beads.

Methods are in some cases limited by destruction of the sample, such that iterative assay steps may not be performed. Iterative reactions, such as sequencing reactions, may be performed on a sample, but upon determining a set of analyte positions through bead binding or sequencing, the sample is often unable to be re-used for a second instance of analyte position determination, in contrast with some approaches disclosed herein.

Against this background, it is an object underlying the present disclosure to provide a method by means of which the disadvantages of the prior art methods can be reduced or even overcome.

### SUMMARY OF THE DISCLOSURE

The present disclosure pertains to novel high resolution multiplex methods and kits for detecting different analytes in a sample beyond the diffraction limit by sequential signal-encoding of said analytes.

Approaches herein allow one to surpass the diffraction limit for detection of the position of adjacent analytes in a sample. This is accomplished in some cases by temporally separating the signal generation of the various analytes, such that signals which would interfere with one another of concurrently generated are generated in processes that are temporally separated so as to facilitate their collection despite the analytes being in close proximity. Signal generation does not require destruction of the sample, such that multiple signals, and even signals of multiple biochemical origins, or even probe-free light imaging of the sample, may be combined with probe-based analyte detection in signal generation. This can be accomplished without sacrificing high-throughput concurrent ‘-omic’ level detection of multiple analytes, up to a substantial portion of a sample's transcriptome or proteome.

In a first aspect two analytic sets are combined in one method to be applied on the same tissue section.

The method is generally characterized by the following steps:
I. Hybridization of target probes for both analytic sets simultaneously before the first run.
II. Tails of the target probes are unique or target identifying for both sets, e.g., 300 tails (for 300 transcripts) are used for the first analytic set and 25 tails (for 25 transcripts) for the second analytic set. This requires or benefits from different or largely different decoder sets for both sets.
III. Both runs generate independent datasets that can be combined in-silico afterwards.

Thereby, it was surprisingly found that the multiplexing capability could be enhanced without increasing optical crowding and spatial overlapping transcripts could be detected which otherwise would be invisible due to the diffraction limit of the microscope.

In a further aspect, embodiments of the disclosure pertain in particular to a multiplex method for detecting different analytes in a sample beyond the diffraction limit by sequential signal-encoding of said analytes, comprising the steps of:
(A1) contacting the sample with a first set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
   (bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(A2) contacting the sample with a second set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
   (bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
wherein (optionally) the number of probes and/or targets of first set of analyte-specific probes according to step A1 (the transcript plexity of A1) is at least 10 times higher than the number of probes and/or targets of the second set of analyte-specific probes according to step A2 (the transcript plexity of A2); and
(B1) contacting the sample with at least a first set of decoding oligonucleotides per analyte, wherein in each set of decoding oligonucleotides for an individual analyte each decoding oligonucleotide of the for the first set of analyte-specific probes according to step A1 comprises:
   (aa) an identifier connector element (t) comprising a nucleotide sequence which is essentially complementary to at least a section of the unique identifier sequence of the identifier element (T) of the corresponding analyte-specific probe set A1, and
   (bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(B2) contacting the sample with at least a second set of decoding oligonucleotides per analyte, wherein in each set of decoding oligonucleotides for an individual analyte of each decoding oligonucleotide for the second set of analyte-specific probes according to step A2 comprises:
   (aa) an identifier connector element (t) comprising a nucleotide sequence which is essentially complementary to at least a section of the unique identifier sequence of the identifier element (T) of the corresponding analyte-specific probe set A2, and
   (bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t);
(C) contacting the sample with at least a set of signal oligonucleotides, each signal oligonucleotide comprising:
   (aa) a translator connector element (C) comprising a nucleotide sequence which is essentially complementary to at least a section of the nucleotide sequence of a translator element (c) comprised in a decoding oligonucleotide, and
   (bb) a signal element.
   (D) Detecting the signal caused by the signal element;
   (E) selectively removing the decoding oligonucleotides and signal oligonucleotides from the sample, thereby essentially maintaining the specific binding of the analyte-specific probes to the analytes to be encoded;
   (F) Performing at least three (3) further cycles comprising steps B) to E) to generate an encoding scheme with a code word per analyte, wherein in particular the last cycle may stop with step (D).

In yet a further aspect, embodiments of this disclosure relate to kits for multiplex analyte encoding beyond the diffraction limit, comprising
(A1) at least a first set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
   (aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(A2) at least a second set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
   (aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
wherein the number of probes and/or targets of first set of analyte-specific probes according to step A1 (the transcript plexity of A1) is at least 10 times higher than the number of probes and/or targets of the second set of analyte-specific probes according to step A2 (the transcript plexity of A2); and
(B) at least one set of decoding oligonucleotides per analyte set A1 and A2, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(C) a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.

In a third aspect, embodiments of this disclosure relate to in vitro methods for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases comprising the use of the multiplex method according to the present disclosure.

In a fourth aspect, embodiments of this disclosure provide in vitro methods for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment, said method comprising the use of the multiplex method according to the present disclosure.

In a fifth aspect, some embodiments of this disclosure relate to optical multiplexing systems suitable for the method according to the present disclosure, comprising at least: at least one reaction vessel for containing the kits or part of the kits according to any one of the claim; a detection unit comprising a microscope, in particular a fluorescence microscope; a camera; a liquid handling device.

In a sixth aspect, some embodiments provide in vitro methods for screening, identifying and/or testing a substance and/or drug comprising:
a) contacting a test sample comprising a sample with a substance and/or drug
b) detecting different analytes in a sample by sequential signal-encoding of said analytes with a method according to the present disclosure.

According to the present disclosure, unique or target specifying tags (identifier) are used per target (e.g., mRNA of one single gene) or for a target group. Groups can be formed to be indicative for a certain identity, process, biological function, or disease (examples: cell type, inflammation, signal processing, cancer).

Importantly, the methods and kits according to the present disclosure leads to a reduction of complexity. Many different probes with different binding sequences share the same (one per target) unique tag. These tags have reduced the sequence complexity (to one per target) and also have predetermined constant properties (e.g., thermodynamic stability).

Advantages of the methods and kits according to the present disclosure are:
a) Full flexibility of the process to determine the identity of the tag, e.g. use more or less signals and/or rounds, varying numbers of fluorophores, number of total signals per tag→lower numbers of targets (e.g. 20) can be identified with high confidence in less rounds (e.g. 4) than a large number of targets (e.g. 100, these need 8 rounds for the same level of confidence), even if in both cases the exact same unique tags are used.
b) All unique tags are used (recycled) in many consecutive rounds of hybridization and all primary probes contribute (provide information about their identity) in every round of identification.
c) As all tags share the same predefined properties (e.g., thermodynamic stability which allows for selective denaturing).

In some advantageous embodiments, the unique tags are design as follow:
No cross-hybridization between all oligonucleotides of the process (probes, decoders, readout), so that all tag sequences are usable together (compatible)
No cross-hybridization between connector elements (bridges) of different unique tags
Stability of hybridization of the unique tags should be in a narrow range: as stable as possible (fast hybridization, that is, short cycle times) but significantly different (in this case less stable) than the primary probe (for differential denaturation, without removing primary probes)

Therefore, the present description pertains in particular to the usage of a set of labeled and unlabeled nucleic acid sequences for specific quantitative and/or spatial detection of different analytes via specific hybridization. The technology allows the discrimination of more different analytes than different detection signals are available. The discrimination is realized via sequential signal-coding of the analytes achieved by several cycles of specific hybridization, detection of signals and selective elution of the hybridized nucleic acid sequences. In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds. The utilization of decoding-oligonucleotides leads to a sequential signal-coding technology that is e.g., more flexible, cheaper, simpler, faster and/or more accurate than other methods.

Furthermore, the present disclosure pertains to the use of improved decoding-oligonucleotides to increase the efficiency of the encoding scheme. The so called “multi-decoders” allows the recruiting of more than just one signal oligonucleotide and therefore can generate new signal types by utilizing the combination of two or more different signal-oligonucleotides without decreasing the brightness of the signals.

Furthermore, due to the use of a first set of analyte-specific probes according to step A1 (the transcript plexity of A1) which can be at least 10 times higher in numbers than the number of probes and/or targets of the second set of analyte-specific probes according to step A2 (the transcript plexity of A2) as well as the use of at least two different sets of decoding oligonucleotides for set A1 and set A2, spatially overlapping targets inside a tissue and or cell culture sample which distance is beyond the diffraction limit can be detected. Further advantages pertain to an improvement of the overall signal to noise ratio, the signal spread (i.e signals of transcripts with higher expression levels can be detected together with lowly expressed genes) and the multiplexing capability without increasing optical crowding.

Before the disclosure is described in detail, it is to be understood that this disclosure is not limited to the particular component parts of the steps of the methods described. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1: Embodiment where the analyte is a nucleic acid, and the probe set comprises oligonucleotides specifically binding to the analyte. The probes comprise a unique identifier sequence allowing hybridization of decoding oligonucleotides.

FIG. 2: Embodiment where the analyte is a protein, and the probe set comprises proteins (here: antibodies) specifically binding to the analyte. The probes comprise a unique identifier sequence allowing hybridization of decoding oligonucleotides.

FIG. 3: Flowchart of the method according to the disclosure.

FIG. 4: Alternative options for the application of decoding and signal oligonucleotides.

FIG. 5: Example for signal encoding of three different nucleic acid sequences by two different signal types and three detection rounds; in this example, the encoding scheme includes error detection.

FIG. 6: Number of generated code words (logarithmic scale) against number of detection cycles.

FIG. 7: Calculated total efficiency of a 5-round encoding scheme based on single step efficiencies.

FIG. 8: Comparison of relative transcript abundances between different experiments.

FIG. 9: Correlation of relative transcript abundances between different experiments.

FIG. 10: Comparison of intercellular distribution of signals.

FIG. 11: Comparison of intracellular distribution of signals.

FIG. 12: Distribution pattern of different cell cycle dependent transcripts.

FIG. 13: Detection of multiple targets using an 8 round code with 2 labels (A and B) and no label (-). The targets 1, 2, 3, 4, 5, 20, and n are represented. The rounds 1, 2, 3, and 8 of the coding scheme are represented. Herein, the blank is part of the code.

FIG. 14: Detection of multiple targets can be performed by an encoding scheme using a detectable marker. The ending scheme may comprise also the “0” as a marker. That means that at a specific position the transcript is not detected. Consequently, the encoding scheme may be represented by the following constructs using only two gene specific probes:
1) With detectable label F: detectable during imaging
2) With detectable label F and quencher Q: not detectable during imaging
3) With quencher Q: not detectable during imaging
4) Without label F: not detectable during imaging
5) Without signaling oligonucleotide: not detectable during imaging
6) With a decoder oligonucleotide that cannot recruit a signaling oligonucleotide
7) Without decoder oligonucleotide: not detectable during imaging

FIG. 15: Possible structures of a multi-decoder. The numbers depict the examples. (A) is the unique identifier sequence, (a) is the corresponding sequence of the decoding oligonucleotide or multi-decoder and (c1) to (c3) are different sequence elements, that specifically bind to different signal oligonucleotides. Examples 2 to 5 show different versions of multi decoders. The order of the different sequence elements as well as the number of signal oligonucleotide binding elements is not fixed. Example 1 shows a normal decoding oligonucleotide since there is only one signal oligonucleotide binding element (c1).

FIG. 16: Example for signal encoding of three different nucleic acid sequences by using multi-decoders and two different signal oligonucleotides creating three different signal types and three detection rounds. In this example, the encoding scheme includes error detection and correction.

FIG. 17: Number of generated codewords (logarithmic scale) against number of detection cycles. The number of codewords for merFISH does not exponentially increase with the number of detection cycles but gets less effective with each added round. In contrast, the number of codewords for intronSeqFISH, the method of the present disclosure without using multi-decoders, the method with multi-decoders increases exponentially. The slope of the curve for the method using multi-decoders is much higher than that of the prior invention, leading to more than 20 000 000 times more code words usable after 20 rounds of detection.

### DETAILED DESCRIPTION OF THE DISCLOSURE

Disclosed herein are novel high-resolution multiplex methods and kits for detecting different analytes in a sample beyond the diffraction limit by sequential signal-encoding of said analytes.

The present disclosure describes the usage of at least two sets of labeled and unlabeled nucleic acid sequences for specific quantitative and/or spatial detection of different analytes via specific hybridization. The technology allows the discrimination of more different analytes than different detection signals are available. The discrimination may be realized via sequential signal-coding of the analytes achieved by several cycles of specific hybridization, detection of signals and selective elution of the hybridized nucleic acid sequences.

In contrast to other state-of-the-art methods, the oligonucleotides providing the detectable signal such as electromagnetic emission are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds.

Furthermore, in contrast to other state-of-the-art methods using already “decoding-oligonucleotides”, at least two different sets of “decoding-oligonucleotides” are used directed to each of the at least two different analytic sets, in order to allow the detection of different subgroups of targets within one analytical run, such that any spatial resolving limitation no longer applies.

The first analytical set is in some cases optimized with respect to specificity, sensitivity, and affinity by the following steps:

The target sequence is scanned, and some or even every position (each base) is used as a starting point to be extended until the predicted hybridization of the complementary part (prospect probe candidate) reaches the minimal required binding stability. After this step, candidates containing homopolymers, repeated motifs or low complexity (e.g., only consist of two different bases) are discarded. The remaining probe candidates are used to find stable binding sites in all other transcripts from the same organism to detect off-target binding. Probe candidates with high affinity to non-targeted sequences are discarded. The final list of candidates is scored and ranked by on-target binding characteristics: Binding on multiple targeted transcript variants increases the score (depending on the transcript annotation class, common and canonical transcript score highest, low evidence transcripts or non-coding isoforms lowest), and a bonus is given if the binding region overlaps the protein coding region of the gene. Finally, highest ranked probe candidates are selected, avoiding large overlaps between probes.

Alternate approaches that result in multiple oligo probes that bind to distinct portions of a common nucleic acid or other analyte target, preferably under common conditions, are also consistent with the disclosure herein, such that probe sets designed through alternate approaches are not excluded from the scope of the present disclosure.

In one aspect of the method of the present invention, the analytical rounds can be arranged consecutively, meaning the detection round(s) of a first analytical set is finished before the detection round(s) of a second analytical set start.

In a second aspect of the method of the present invention, the analytical rounds can be arranged interleaved, meaning the detection rounds of a first analytical set and a second analytical set alternate in a certain pattern, e.g.:
Detection round 1=1st round of detection of first analytical set.
Detection round 2=1st round of detection of second analytical set.
Detection round 3=2nd round of detection of first analytical set.
Detection round 4=2nd round of detection of second analytical set
Etc.

The utilization of decoding-oligonucleotides leads to a sequential signal-coding technology that is more flexible, cheaper, simpler, faster and/or more accurate than other methods and allows a resolution beyond the diffraction limit of optical microscopes, without requiring fluorophore-labeled analyte specific probes.

It is one advantage of the present invention to detect a larger number of different targets (e.g., transcripts, genes, proteins or other analytes) with fewer detection cycles, or fewer cycles per analyte detected, as compared to the prior art.

### A. Definitions

According to the present disclosure an “analytical set” is a set of probes specific for a subgroup of targets. An analytical set of probes is often applied to a sample and decoded as a group. Preferably the targets within a first analytical set directed to a subgroup of targets which do not usually show a high spatial overlap, whereas a second analytical set directed to a subgroup of targets which may spatially overlap with the first subgroup and/or may add additional information which allows a further differentiation of the signals found with the first analytical set. For example, a first analytical set may be directed to known activating mutations in promotor-structures with a high prevalence for cancer. The second analytical set may be directed to genes associated with cancer development when over-expressed. Thus, a colocalization of signals detected with the first and the second analytical set may indicate activated promotors in genes associated with cancer.

In yet another embodiment fusion genes may be detected by this method. Fusion genes, and the fusion proteins that come from them, may occur naturally in the body when part of the DNA from one chromosome moves to another chromosome. Fusion proteins produced by this change may lead to the development of some types of cancer. The disclosed method allows the detection of co-localization other normally distinct gene loci and therefore the detection of certain cancer types, e.g. cancers which are the result of the fusion of the following genes: Adenoid cystic carcinoma as result of MYB-NFIB and/or NFIB-HMGA2 fusion; mucoepidermoid carcinoma as a result of MECT1-MAL2 fusion; follicular thyroid carcinoma as a result of PAX87-PPARG fusion; breast carcinoma as a result of ETV6-NTRK3 and/or FGFR3-AFF3 and/or FGFR2-CASP7 and/or FGFR2-CCDC6 and/or ERLINK2-FGFR1 fusion; Ewing sarcoma as a result of EWSR1-FLI1 fusion; Small round cell tumors of bone BCOR-CCNB3 fusion; Synovial sarcoma as a result of SS18-SSX1 and/or SS18-SSX2 fusion; glioblastoma multiforme as a result of FGFR3-TACC3 and/or FGFR1-TACC1 fusion; pilocytic astrocytoma as a result of KIAA1967-BRAF fusion; lung cancer as a result of EML4-ALK and/or FGFR3-TACC3 and/or FGFR3-KIAA1967 and/or BAG4-FGFR1 fusion; clear cell renal cell carcinoma as a result of SFP!-TFE3 and/or TFG-GPR128 fusion; bladder cancer as a result of FGFR3-TACC3 and/or FGFr3-BAIAP2L1 fusion; prostate cancer as a result of TMPRSS2-ERG/ETV1/ETV4 and/or SLC45A3-FGFR2 fusion; ovarian cancer as a result of ESRRA-C11orf20 fusion; and/or colorectal cancer as a result of PTPRK-RSPO3 and/or EIF3E-RSPO2 fusion. Of course, also other combinations can be envisaged, for example tissue-type-specific probes with cancer markers, proto-oncogenic targets in different combinations, etc.

In one embodiment, a first analytical set may be directed to known transcripts of a certain gene. The second analytical set may be directed to another set of transcripts of the same gene to get information about differential splicing. Thus, a colocalization signals detected with the first and the second analytical set may detect differentially spliced exons, different polyadenylation sites, differential degradation products or other differential variation observed among transcripts of the same gene.

According to the present disclosure an “analyte” is the subject to be specifically, uniquely or distinctly detected as being present or absent in a sample and, in case of its presence, to encode it. It can be any kind of entity, including a protein, polypeptide, or a nucleic acid molecule (e.g. RNA, PNA or DNA) or even a small molecule of interest. The analyte provides at least one site for specific binding with analyte-specific probes. Sometimes herein the term “analyte” is replaced by “target”. An “analyte” according to the disclosure incudes a complex of subjects, e.g., at least two individual nucleic acid, protein or peptides molecules. In some cases an “analyte” excludes a chromosome, while alternately in some cases an “analyte” excludes DNA.

In some embodiments, an analyte may be a “coding sequence”, “encoding sequence”, “structural nucleotide sequence” or “structural nucleic acid molecule” which refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, RNA, EST and recombinant nucleotide sequences.

A “sample” as referred to herein is a composition in liquid or in many referred embodiments in solid form, suspected of comprising the analytes to be encoded or for which an analyte or a set of analytes is to be assayed. In particular, the sample is a biological sample, preferably comprising biological tissue, further preferably comprising biological entities such as cells, viruses, and/or extracts and/or part of cells or morphological structures. For example, the cell is a prokaryotic cell or a eukaryotic cell, in particular a mammalian cell, in particular a human cell. In some embodiments, the biological tissue, biological cells, extracts and/or part of cells are fixed. In particular, the analytes are fixed in a permeabilized sample, such as a cell-containing sample. The sample may also be fixed to a surface, and is preferably prepared such that two-dimensional positional information is preserved.

As used in the present disclosure, “cell”, “cell line”, and “cell culture” can be used interchangeably and all such designations include progeny. Thus, the words “transformants” or “transformed cells” include the primary subject cell and cultures derived therefrom without regard for the number of transfers. It is also understood that all progenies may not be precisely identical in DNA content, due to deliberate or inadvertent mutations. Mutant progeny that have the same functionality as screened for in the originally transformed cell are included.

An “encoding scheme” may describe a set of “code words” that are associated with the analytes to be detected. Each “code word” refers to one or a few of the analytes and can be distinguished from some or all other “code words”. A code word hereby is a sequence of signs provided by the detection cycles of the method. A sign within a “code word” is a detectable signal or the absence of a signal. A “code word” does not need to comprise of all different signals used in the method. The number of signs in a “code word” is defined by the number of detection cycles. Code words are the product of the iterative assaying for a detection signal over multiple steps, while the order of ‘letters’ in the code words is determined by the selection of decoding oligos used in each iterative step of the iterative assaying.

An “oligonucleotide” as used herein, refers to s short nucleic acid molecule, such as DNA, PNA, LNA or RNA. The length of the oligonucleotides is within the range 4-200 nucleotides (nt), preferably 6-80 nt, more preferably 8-60 nt, more preferably 10-50 nt, more preferably 12 to 35 depending on the number of consecutive sequence elements. The nucleic acid molecule can be fully or partially single-stranded. The oligonucleotides may be linear or may comprise hairpin or loop structures. The oligonucleotides may comprise modifications such as biotin, labeling moieties, blocking moieties, or other modifications.

The “analyte-specific probe” consists of at least two elements, namely the so-called binding element(S) which specifically interacts with one of the analytes, and a so-called identifier element (T) comprising the ‘unique identifier sequence’. The binding element(S) may be a nucleic acid such as a hybridization sequence or an aptamer, or a peptidic structure such as an antibody. The identifier element (T) may be unique to a particular target, or may be sufficiently distinct enough to allow identification of a targeted analyte to the exclusion of one or more up to all other analytes in a sample or assayed for in a detection round, or assayed for in an analytical set.

Also a “probe” may comprise or consist of at least two elements, namely the so-called binding element(S) which specifically interacts with one of the analytes, and a so-called identifier element (T) comprising the ‘unique identifier sequence’. The binding element(S) may be a nucleic acid such as a hybridization sequence or an aptamer, or a peptidic structure such as an antibody.

In particular, in some embodiments the binding element(S) comprises moieties which are affinity moieties from affinity substances or affinity substances in their entirety selected from the group consisting of antibodies, antibody fragments, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and their derivatives, mutants or combinations thereof. In further advantageous embodiments, the antibody fragment is a Fab, an scFv; a single domain, or a fragment thereof, a bis scFv, Fab2, Fab3, minibody, maxibody, diabody, triabody, tetrabody or tandab, in particular a single-chain variable fragment (scFv).

The “unique identifier sequence” as comprised by the analyte-specific probe is unique in its sequence compared to other unique identifiers. “Unique” in this context means that it specifically identifies only one analyte, such as Cyclin A, Cyclin D, Cyclin E etc., or, alternatively, it specifically identifies only a group of analytes, independently whether the group of analytes comprises a gene family or not, or that it identifies a particular analyte to the exclusion of one or more alternative analytes, up to and in many cases excluding all other analytes in an analytical set. Therefore, the analyte or a group of analytes to be encoded by this unique identifier can be distinguished from one or more, some or in many cases all other analytes or groups of analytes that are to be encoded based on the unique identifier sequence of the identifier element (T). Or, in other words, there is only one ‘unique identifier sequence’ for a particular analyte or a group of analytes in an analysis set, but not more than one, in some cases not even two. Due to the uniqueness of the unique identifier sequence the identifier element (T) often hybridizes to exactly one type of decoding oligonucleotides in a sample or in a given analysis set. The length of the unique identifier sequence is within the range 8-60 nt, preferably 12-40 nt, more preferably 14-20 nt, depending on the number of analytes encoded and the stability of interaction needed. Alternately, in some cases a unique identifier sequence can be distinguished for at least one other analyte or group of analytes that are to be encoded.

In one embodiment modified oligonucleotides may be used to crosslink the hybridized elements permanently (e.g. covalently), for example to establish a permanent or enduring connection between analyte specific probe and decoding oligonucleotide(s); between decoding oligonucleotide(s) and signal oligonucleotide(s); and/or analyte specific probe and decoding oligonucleotide(s) and signal oligonucleotide(s).

A unique identifier may be a sequence element of the analyte-specific probe, attached directly or by a linker, a covalent bond or high affinity binding modes, e.g. antibody-antigen interaction, streptavidin-biotin interaction etc. It is understood that the term “analyte specific probe” includes a plurality of probes which may differ in their binding elements(S) in a way that each probe binds to the same analyte but possibly to different parts thereof, for instance to different (e.g. neighboring) or overlapping sections of the nucleotide sequence comprised by the nucleic acid molecule to be encoded. However, each of the plurality of the probes comprises the same identifier element (T). A “bipartite labeling probe” comprises a binding sequence capable of hybridizing the analyte and a binding probe sequence capable of binding a detectable signal molecule like a fluorophore or a nucleic acid sequence comprising a fluorophore.

A “decoding oligonucleotide” or an “adapter” or “adapter segment” comprises at least two sequence elements or one sequence element and one moiety that binds to a unique or specific portion of a primary analyte binding probe. One sequence element that can specifically bind to a unique identifier sequence, referred to as an “identifier connector element”(t) or “first connector element” (t), and a second sequence element specifically binding to a signal oligonucleotide, referred to as “translator element” (c). The length of the sequence elements is within the range 8-60 nt, preferably 12-40 nt, more preferably 14-20 nt, de-pending on the number of analytes to be encoded, the stability of interaction needed and the number of different signal oligonucleotides used. The length of the two sequence elements may or may not be the same.

In this respect it is important to note, that the decoding-oligonucleotide may be adapted to the specific needs for the respective test or diagnosis. For example, a patient sample may be first screened for general tumor markers, and then in consecutive rounds for markers of specific tumor subtypes (e.g. breast cancer) or even molecular subtypes of a specific cancer (e.g. HER2-negative vs. HER2-positive). In those cases, not only the analytical set, but also the decoding-oligonucleotides may be designed to provide a specific pattern, which allows to maximize the information gained from a sample.

In some advantageous embodiments, the decoding oligonucleotide in the kits and/or methods of the present disclosure may be a “multi-decoder”. A “multi-decoder” is a decoding oligonucleotide that comprises at least three sequence elements. One sequence element (the identifier connector element (t)) can specifically bind to a unique identifier sequence (identifier element (T)) and at least two other sequence elements (translator elements (c)) specifically bind different signal oligonucleotides (each of these sequence elements specifically binds a signal oligonucleotide that differs to all other signal oligonucleotides recruited by other elements of the multi-decoder). The length of the sequence elements is within the range 8-60 nt, preferably 12-40 nt, more preferably 14-20 nt, depending on the number of analytes detected, the stability needed, and the number of different signal oligonucleotides used. The length of the sequence elements may or may not be the same.

Therefore, in some advantageous embodiments, the decoding oligonucleotide is a multi-decoder comprising
an identifier connector element (t) comprising a nucleotide sequence which is essentially complementary to at least a section of the unique identifier sequence of the identifier element (T) of the corresponding analyte-specific probe set, and
at least two translator elements (c) comprising each a nucleotide sequence allowing a specific hybridization of a different signal oligonucleotide.

Therefore, the first translator element binds a different signal oligonucleotide as the second translator element. In particular, the signal oligonucleotides differ in the signal element comprised in the signal oligonucleotide, e.g., in the kind of the fluorophore.

A “signal oligonucleotide” or a “reporter” as used herein comprises at least two elements, a so-called “translator connector element” (C) or “second connector element” (C) having a nucleotide sequence specifically hybridizable to at least a section of the nucleotide sequence of the translator element (c) of the decoding oligonucleotide, and a “signal element” which provides a detectable signal. This element can either actively generate a detectable signal or provide such a signal via manipulation, e.g. fluorescent excitation. Typical signal elements are, for example, enzymes that catalyze a detectable reaction, fluorophores, radioactive elements or dyes. A signal from a signal element may form a ‘letter’ of a sequentially generated analyte identifying ‘codeword.’

A “set” refers to a plurality of moieties or subjects, e.g. analyte-specific probes or decoding oligonucleotides, whether the individual members of said plurality are identical or different from each other. In an analyte specific probe set, the analyte specific probes are identical in the identifier element (T) but may comprise a different binding element(S) for specifically interacting with the same analyte but for specifically interacting with different sub-structures of the same analyte to be encoded. “Selective denaturation” may be the process of eliminating bound decoding oligonucleotides and signal oligonucleotides with highest efficiency while at the same time the target specific probes have to stay hybridized with the highest efficiency.

The total efficiency of these two combined events may to be at least 0.22 for two detection cycles, at least 0.37 for three detection cycles, at least 0.47 for four detection cycles, at least 0.55 for five detection cycles, at least 0.61 for six detection cycles, at least 0.65 for seven detection cycles, at least 0.69 for eight detection cycles, at least 0.72 for nine detection cycles and at least 0.74 for 10 detection cycles, at least 0.76 for 11 detection cycles and at least 0.78 for 12 detection cycles.

In an embodiment of the disclosure a single set refers to a plurality of oligonucleotides: An “analyte specific probe set” refers to a plurality of moieties or subjects, e.g. analyte-specific probes that are different from each other and bind to independent regions of the analyte. A single analyte specific probe set is further characterized by the same unique identifier.

A “decoding oligonucleotide set” refers to a plurality of decoding oligonucleotides specific for a certain unique identifier needed to realize the encoding independent of the length of the code word. Each and all of the decoding oligonucleotides included in a “decoding oligonucleotide set” bind to the same unique identifier element (T) of the analyte-specific probe.

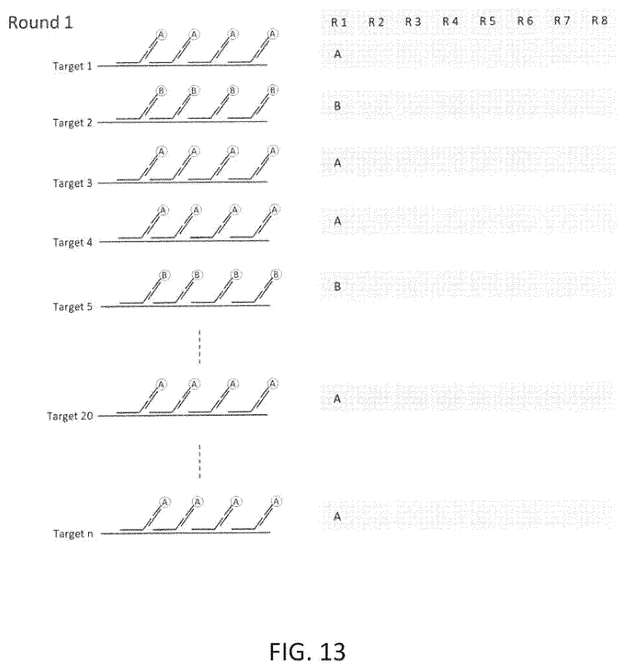

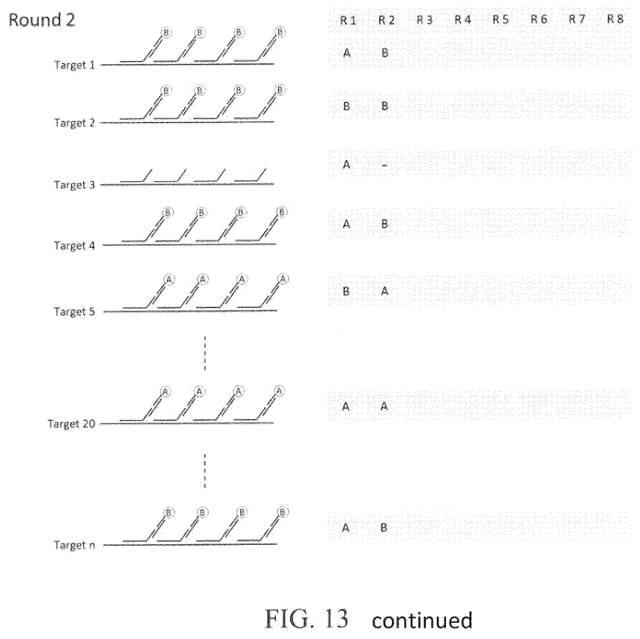

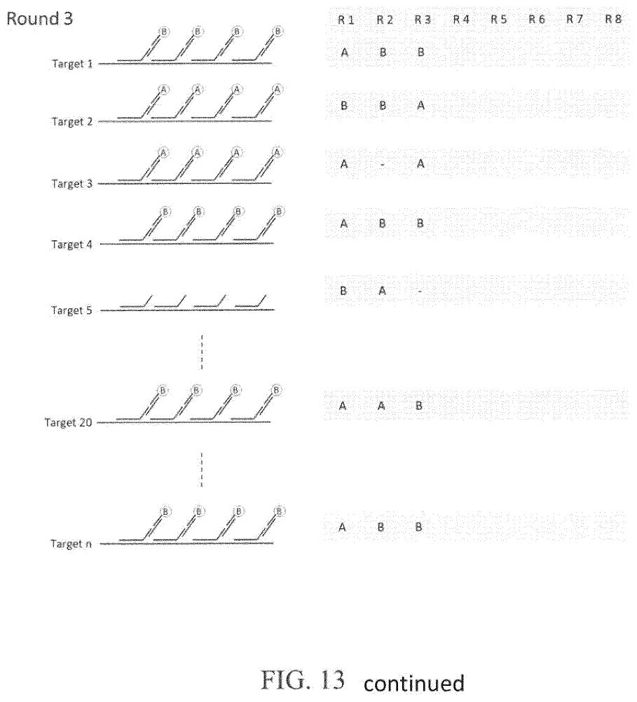

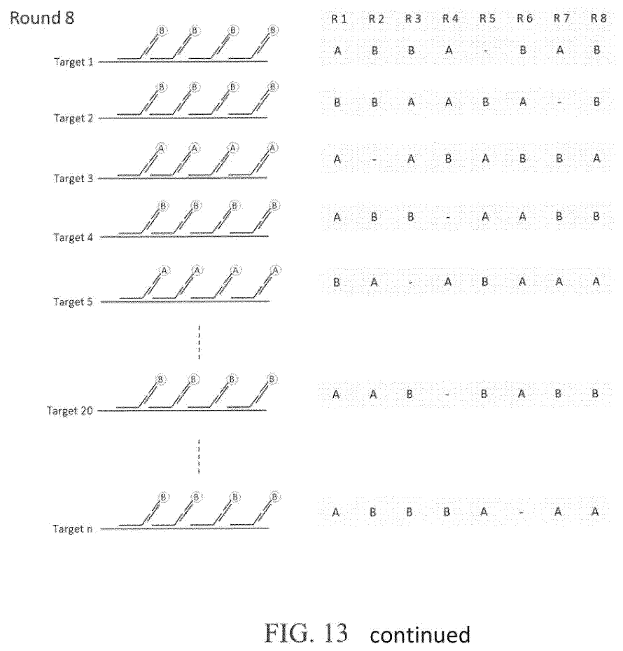

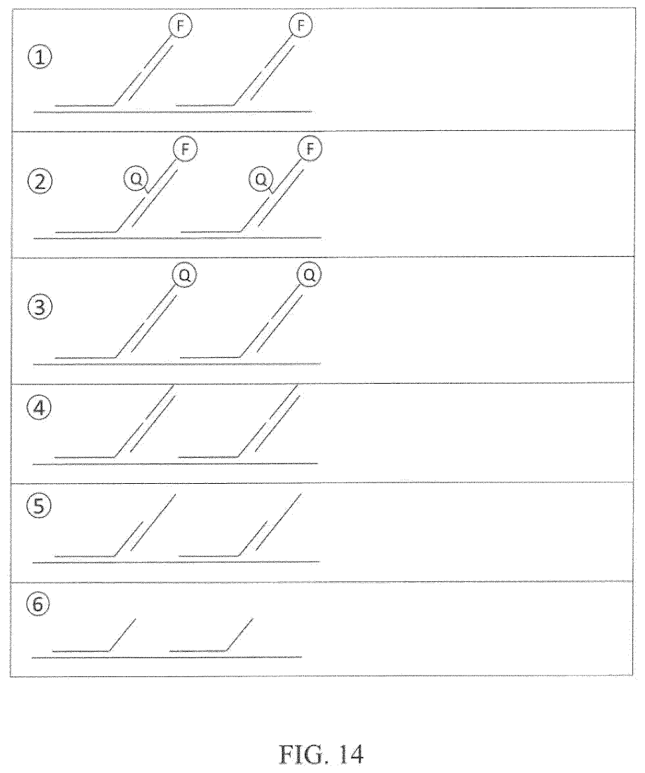

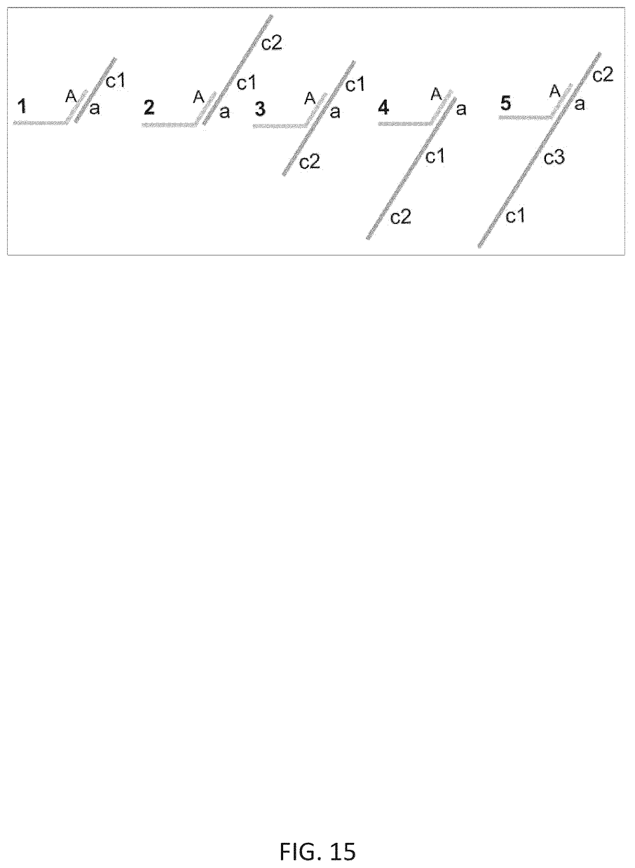

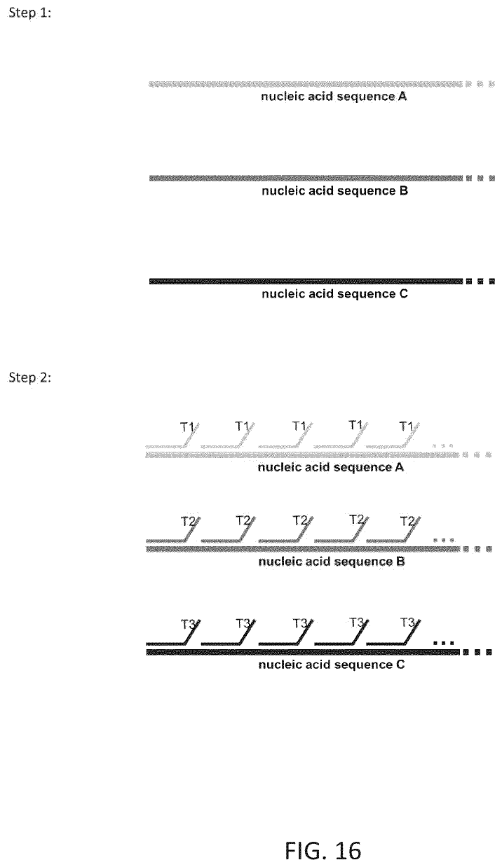

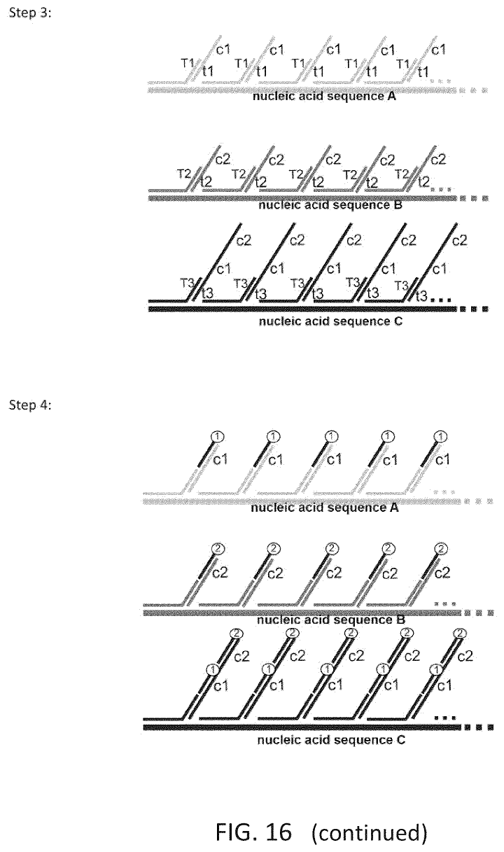

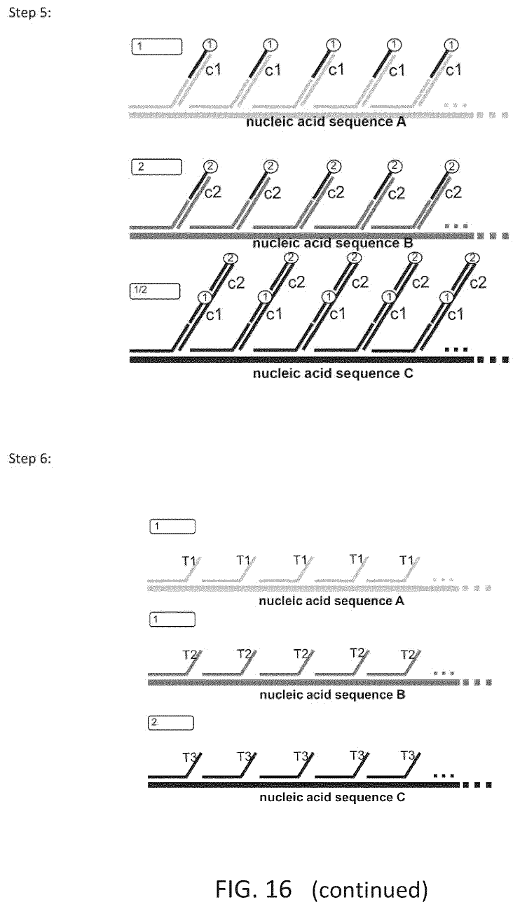

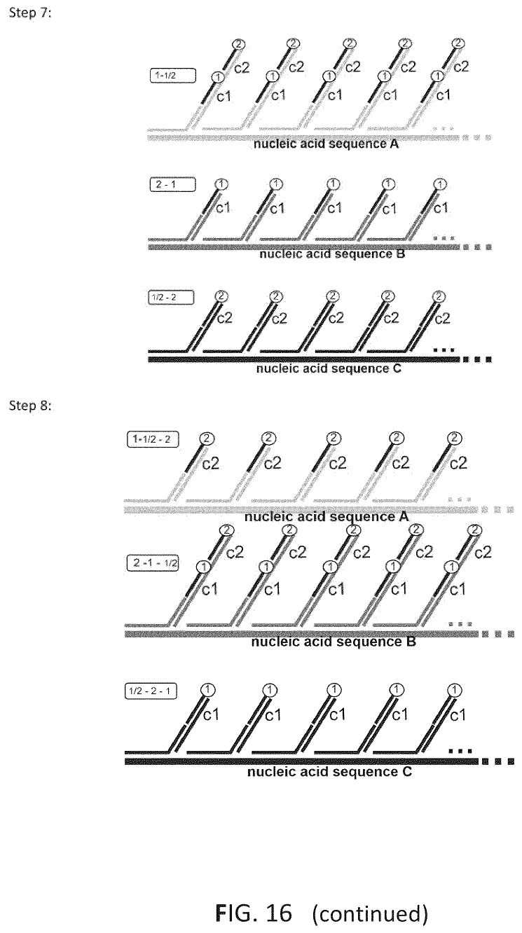

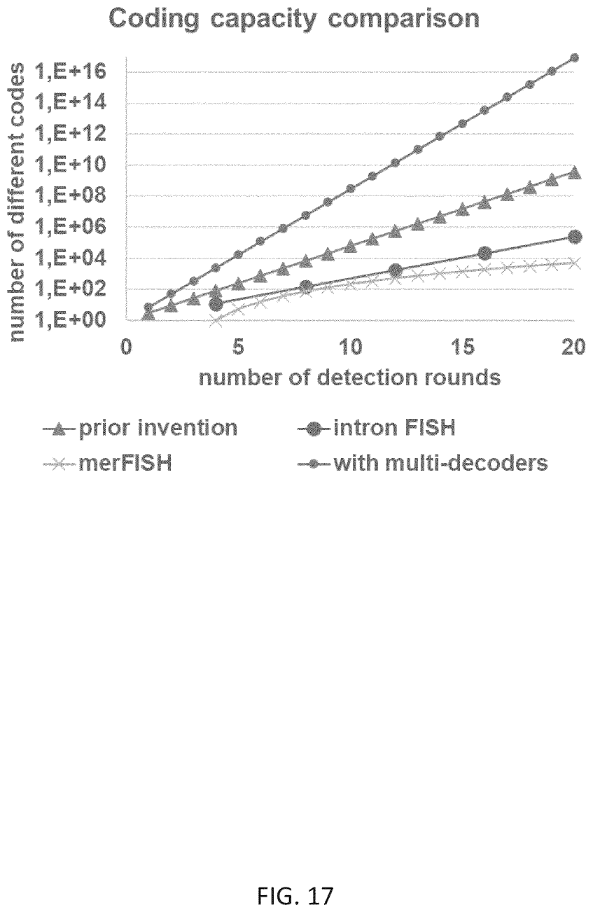

In certain embodiments, this pattern of binding or hybridization of the decoding oligonucleotides may be converted into a “codeword.” For example, the codewords could be also “101” and “110” for an analyte, where a value of 1 represents binding and a value of 0 represents no binding. The codewords may also have longer lengths in other embodiments (see FIG. 13). A codeword can be directly related to a specific unique identifier sequence of a analyte-specific probe. Accordingly, different analyte-specific probes may match certain codewords, which can then be used to identify the different analytes of the analyte-specific probe based on the binding patterns of the decoding oligonucleotide. However, if no binding is evident, then the codeword would be “000” in this example.

The values in each codeword can also be assigned in different fashions in some embodiments. For example, a value of 0 could represent binding while a value of 1 represents no binding. Similarly, a value of 1 could represent binding of a secondary nucleic acid probe with one type of signaling entity while a value of 0 could represent binding of a secondary nucleic acid probe with another type of distinguishable signaling entity. These signaling entities could be distinguished, for example, via different colors of fluorescence. In some cases, values in codewords need not be confined to 0 and 1. The values could also be drawn from larger alphabets, such as ternary (e.g., 0, 1, and 2) or quaternary (e.g., 0, 1, 2, and 3) systems. Each different value could, for example, be represented by a different distinguishable signaling entity, including (in some cases) one value that may be represented by the absence of signal.

The codewords for each analyte may be assigned sequentially, or may be assigned at random. For instance, a first analyte may be assigned to 101, while a second nucleic acid target may be assigned to 110. In addition, in some embodiments, the codewords may be assigned using an error-detection system or an error-correcting system, such as a Hamming system, a Golay code, or an extended Hamming system (or a SECDED system, that is, single error correction, double error detection). Generally speaking, such systems can be used to identify where errors have occurred, and in some cases, such systems can also be used to correct the errors and determine what the correct codeword should have been. For example, a codeword such as 001 may be detected as invalid and corrected using such a system to 101, e.g., if 001 is not previously assigned to a different target sequence. A variety of different error-correcting codes can be used, many of which have previously been developed for use within the computer industry; however, such error-correcting systems have not typically been used within biological systems. For example, one or more digits of the code-word could be reserved for an internal control (e.g., checksum). A similar approach is for example used in case of credit card numbers, but not known for biological systems. Additional examples of such error-correcting codes are discussed in more detail below.

Thus, in one embodiment an error-correction system is integrated in the binding element(S) and/or identifier element (T) and/or identifier connector element (t) and/or translator element (c) and/or translator connector element (C) and/or signal element.

“Essentially complementary” means, when referring to two nucleotide sequences, that both sequences can specifically hybridize to each other under stringent conditions, thereby forming a hybrid nucleic acid molecule with a sense and an antisense strand connected to each other via hydrogen bonds (Watson-and-Crick base pairs). “Essentially complementary” includes not only perfect base-pairing along the entire strands or the entire intended binding region along the strands, such as perfect complementary sequences but also imperfect complementary sequences which, however, still have the capability to hybridize to each other under stringent conditions. Among experts it is well accepted that an “essentially complementary” sequence has at least 88% sequence identity to a fully or perfectly complementary sequence.

“Percent sequence identity” or “percent identity” in turn means that a sequence is compared to a claimed or described sequence after alignment of the sequence to be compared (the “Compared Sequence”) with the described or claimed sequence (the “Reference Sequence”). The percent identity is then determined, for example according to the following formula: percent identity=100 [1−(C/R)] wherein C is the number of differences between the Reference Sequence and the Compared Sequence over the length of alignment between the Reference Sequence and the Compared Sequence, wherein
(i) each base or amino acid in the Reference Sequence that does not have a corresponding aligned base or amino acid in the Compared Sequence and
(ii) each gap in the Reference Sequence and
(iii) each aligned base or amino acid in the Reference Sequence that is different from an aligned base or amino acid in the Compared Sequence, constitutes a difference and (iv) the alignment has to start at position 1 of the aligned sequences;
and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity, then the Compared Sequence has the specified minimum percent identity to the Reference Sequence even though alignments may exist in which the herein above calculated percent identity is less than the specified percent identity.

In the “incubation” steps as understood herein the respective moieties or subjects such as probes or oligonucleotide, are brought into contact with each other under conditions well known to the skilled person allowing a specific binding or hybridization reaction, e.g. pH, temperature, salt conditions etc. Such steps may therefore, be preferably carried out in a liquid environment such as a buffer system which is well known in the art.

The “removing” steps according to the disclosure may include the washing away of the moieties or subjects to be removed such as the probes or oligonucleotides by certain conditions, e.g. pH, temperature, salt conditions etc., as known in the art. A benefit of many embodiments herein is that annealing, incubation and removing are not detrimental or not destructive to sample stability, such that multiple rounds of each step may be performed, with more than one analysis set, before sample degradation precludes additional analysis. In some cases an analysis protocol leaves a sample intact such that it may be imaged subsequent to analysis set codeword determination, allowing the codewords or the identity of the encoded analytes to be mapped onto an image of the sample.

It is understood that in an embodiment of the method according to the present disclosure a plurality of analytes can be encoded. This requires the use of different sets of analyte-specific probes in step (1). The analyte-specific probes of a particular set differ from the analyte-specific probes of another set. This means that the analyte-specific probes of set 1 bind to analyte 1, the analyte-specific probes of set 2 bind to analyte 2, the analyte-specific probes of set 3 bind to analyte 3, etc. In this embodiment also the use of different sets of decoding oligonucleotides is required in the methods according to the present disclosure.

The decoding oligonucleotides of a particular set differ from the decoding oligonucleotides of another set. This means, the decoding oligonucleotides of set 1 bind to the analyte-specific probes of above set 1 of analyte-specific probes, the decoding oligonucleotides of set 2 bind to the analyte-specific probes of above set 2 of analyte-specific probes, the decoding oligonucleotides of set 3 bind to the analyte-specific probes of above set 3 of analyte-specific probes, etc.

In this embodiment where a plurality of analytes is to be encoded the different sets of analyte-specific probes may be provided as a premixture of different sets of analyte-specific probes and/or the different sets of decoding oligonucleotides may be provided as a premixture of different sets of decoding oligonucleotides. Each mixture may be contained in a single vial. Alternatively, the different sets of analyte-specific probes and/or the different sets of decoding oligonucleotides may be provided in consecutive steps (as outlined before).

A “kit” is a combination of individual elements useful for carrying out the use and/or method of the disclosure, wherein the elements are optimized for use together in the methods. The kits may also contain additional reagents, chemicals, buffers, reaction vials etc. which may be useful for carrying out the method according to the disclosure. Such kits unify all essential elements required to work the method according to the disclosure, thus minimizing the risk of errors. Therefore, such kits also allow semi-skilled laboratory staff to perform the method according to the present disclosure.

In yet another aspect the kit may be designed for the use of technical laymen, for examples in environments which necessitate quick analysis of samples for certain markers, such as airports, border control, customs control, personalized medicine, self-diagnosis at home, etc. Thus, the invention also encompasses such uses which do not necessitate the handling of a skilled personal, but only fundamentally trained personal following step-by-step instructions and “ready-to-use”-kits.

The term “quencher” or “quencher dye” or “quencher molecule” refers to a dye or an equivalent molecule, such as nucleoside guanosine (G) or 2′-deoxyguanosine (dG), which is capable of reducing the fluorescence of a fluorescent reporter dye or donor dye. A quencher dye may be a fluorescent dye or non-fluorescent dye. When the quencher is a fluorescent dye, its fluorescence wavelength is typically substantially different from that of the reporter dye and the quencher fluorescence is usually not monitored during an assay. Some embodiments of the present disclosure disclose signal oligonucleotides comprising a quencher and/or a quencher in combination with a signal element (see FIG. 14), and therefore the signal oligonucleotides is not detectable during imaging.

Quenching and other optical effects can also be used in order to identify co-localization of certain signals. For example, the co-localization of a green and red signal may result in a yellow signal, thereby emphasizing a certain maximal distance between both signals. Thus, another level of information can be added by the choice of the fluorescent reporter dye(s).

As used herein, the term “about” a number refers to a range spanning from 10% less than to 10% greater than that number, while “about” a range refers to an extended range spanning from 10% below the lower limit listed for the range to 105 above the upper limit listed for the range.

### B. Aspects of the Disclosure

Indirect, sequential signal-encoding approaches have a number of advantages over approaches in the art. Rather than using a single labelled probe specific for each target, one uses a combination of commonly labelled target analyte binding probes, decoding probes and signal probes to generate a signal at a target analyte. By iteratively removing and removing the decoding probes and the signal probes, one may generate a temporal series of signals, or “code word” indicative of a target analyte. This is done by controlling the order with which decoding probes corresponding to the target analyte probes for a given target, so as to generate a known series of fluorescence signals unique to or specific to or indicative of the target analyte to which the target binding probes are directed. This code word may be detected and decoded to identify the position of the target analyte at a position in a sample. Decoding oligos and signal oligos may be produced in bulk rather than being synthesized specifically for any particular reaction. Target analyte specific code words are generated using only one or two fluorophores, such that the diversity of targets to be detected far surpasses the diversity of reagents, including fluorophores or probes. Unlike approaches in the art, the indirect sequential labelling approaches allow one to assay a number of analytes that is not limited by the number of unique probes available. One may access potentially the full diversity of genome, transcriptome, proteome or metabolome data in a cell, tissue, organ or organs in a single reaction, providing an unprecedented amount of spatial molecular information.

This efficiency and massive scale, however, does not come without challenges. In particular, if target analytes are positioned such that the distance between them is beyond (that is, closer than) the diffraction limit for the wavelength of the signal generated by the signal probe, the distinct known series of fluorescence signals for the two distinct adjacent target analytes will overlap, such that they cannot be independently detected.

The disclosure herein addresses this challenge, enabling one to detect target analytes situated such that the distance between them is beyond (that is, closer than) the diffraction limit for the wavelength of the signal generated by the signal probe, in addition to conveying other benefits. Accordingly, the disclosure herein relates not only to sequential signal encoding analyte detection, but to general approaches for large scale detection of analytes that are situated in a sample at a distance which, through the use of other approaches, is less than a resolution threshold necessary to distinguish them. Closely positioned target analytes are not a rare or abstract hypothetical issue. A number of oncogenic genomic translocations, for example, result in genes or gene segments being brought into proximity so as to generate chimeric transcripts encoding chimeric proteins associated with particular cancer outcomes. Gene fusion events that generate a chimeric protein are causative for several cancer types, accounting for approximately 20% of tumors overall (Mitelman, F., Johansson, B., & Mertens, F. (2007): The impact of translocations and gene fusions on cancer causation. Nature Reviews. Cancer, 7 (4), 233-245.). Detection of RNA fusions has facilitated the molecular characterization and diagnosis of various tumors (reviewed by Neckles, C, Sundara Rajan, S, Caplen, N J. (2020): Fusion transcripts: Unexploited vulnerabilities in cancer? WIREs RNA; 11: e1562. https://doi.org/10.1002/wrna.1562). The recent approval of molecules that target oncogenic fusion transcripts for degradation suggests that these are promising therapeutic targets. However, the inter- and intra-tumoral diversity of oncogenic fusion transcripts needs to be understood in more detail, ideally on the cellular level or even with subcellular resolution. An incomplete list of examples of oncogenic fusion genes or translocations include BRC-ABL, TEL-AML, AML1-ETO, BCAM-AKT2, and TMPRSS2-ERG.

Similarly, protein-RNA colocalization or protein-protein colocalization into complexes can have dramatic physiological consequences. Colocalization of FK-506 Binding Protein to TOR complexes mediated by rapamycin, for example, has a large impact on TOR complex activity and the myriad cellular processes that it regulates.

Detecting these events, either as their genomic loci, their transcript distribution patterns or their protein distribution patters often requires detecting each portion of the chimeric fusion product separately, and assaying for their colocalization. If, however, their colocalization results in the distance between the targets being beyond (that is, closer than) the diffraction limit for the wavelength of the signal generated by the signal probe, then detection efforts may fail. Furthermore, most approaches require that the events be detected through a targeted, micro-scale approach, rather than incorporating their detection into untargeted, tissue-wide or transcriptome or proteome-wide assays.

For example, gene fusion transcripts may be detected or inferred through hybridization of transcript-specific probes having unique labels. These approaches are effective if one is interested in a particular fusion event in isolation, and one is willing to exhaust an entire sample slice to obtain this information. However, the cost and technical challenge of designing and uniquely labelling each probe with a distinct wavelength-emitting fluorophore quickly limits the ability of traditional FISH approaches to incorporate colocalization assays into transcriptome-wide data gathering, and it precludes data gathering that may uncover novel or untargeted gene or transcript fusion events.

Accordingly, disclosed herein are approaches for analyte colocalization sufficient to detect colocalized analytes as part of the detection of at least 10, 20, 50, 100, 200, 500, 1,000, 2,000, 5,000, 10,000, 20,000 or more than 20,000 analytes. The approaches are in some cases untargeted in that they are not directed to analytes suspected of colocalization to the exclusion or neglect of other analytes in a sample. Detection of analyte colocalization is in some cases effected without analyte specific fluorophores, such that probes used to detect the colocalized analytes are also used in the detection of other analytes.

Positionally-barcoded bead-based approaches are similarly challenged in their ability to resolve issues related to transcript or other analyte colocalization. These approaches rely upon annealing and amplification of transcripts or other nucleic acids on positionally barcoded beads, such that the transcripts or other nucleic acids attached to the beads share a common barcode indicative of their position on a sample. These approaches may interface readily with existing sequencing technology.

However, the level of resolution in these approaches is limited by the size of the bead on which the target analytes are annealed. The level of resolution for approaches using, say, a 10 um diameter bead cannot surpass the size of the bead. As these approaches do not use beads of a size near the diffraction limit of the wavelength of detection, long before the diffraction limit of the wavelength of detection is met, resolution is limited by the size of the bead.

Accordingly, disclosed herein are approaches for analyte colocalization sufficient to distinguish superimposed or adjacent analytes from analytes that are not colocalized but occur within20 um of one another, within 10 um of one another, within 5 um of one another, within 2 um of one another, or within 1 um of one another. Similarly, disclosed herein are approaches for analyte colocalization having a level of resolution of less than 10 um, less than 5 um, less than 2 um, or less than 1 um. Similarly, in some cases sequencing such as bead based sequencing may be combined with the approaches herein, such that transcript sequences are obtained while analyte localization is determined at a level of resolution that is beyond (that is, smaller than) the size of the beads.

Through practice of the disclosure herein, one is able to detect target analytes positioned such that the distance between them is beyond (that is, closer than) the diffraction limit for the wavelength of the signal generated by the signal probe used. In some cases, these approaches use indirect, sequential signal-encoding, alone or in combination with traditional FISH based fluorophore detection or with sequencing based detection methods such as those having a resolution limited by bead size or diffraction limit.

Some approaches herein are directed to nucleic acid detection, such as RNA transcripts, such as RNA transcripts that may be colocalized in physical proximity, or even colocalized due to sharing a common phosphodiester backbone due to their being transcribed from a translocated mutant locus. Putative colocalized transcripts are detected in separate probe sets, assayed either successively or through an interleaved detection scheme, such that in either case signals from the putative colocalized transcripts are not concurrently obtained. This detection process need not be targeted, however, such that many (at least 10, 20, 50, 100, 200, 500, 1,000, 2,000, 5,000, 10,000, 20,000) transcripts may be assayed for in a first probe set and in a second probe set.

In some cases probe sets are selected to have differential densities of targets, such that a first probe set exhibits a higher or lower target density or number of target analytes. Some second probe sets are tailored to target analytes of interest in light of first probe set results. For example, if a first probe set identifies a transcript that is frequently involved in transcript concatemers, then in some cases the concatemer partner transcript probe is included in a second probe set. Similarly, some probe sets are selected such that known frequently colocalized transcripts or other analytes are probed for in distinct probe sets, such that their signals are not generated concurrently. Thus, for example, for one or more of the fusions listed below, probe sets targeting members of a particular fusion are found in distinct probe sets in some embodiment: MYB-NFIB NFIB-HMGA2 MECT1-MAL2 PAX87-PPARG ETV6-NTRK3 FGFR3-AFF3 FGFR2-CASP7 FGFR2-CCDC6 ERLINK2-FGFR1 EWSR1-FLI1 BCOR-CCNB3 SS18-SSX1 SS18-SSX2 FGFR3-TACC3 FGFR1-TACC1 KIAA1967-BRAF EML4-ALK FGFR3-TACC3 FGFR3-KIAA1967 BAG4-FGFR1 SFP1-TFE3 TFG-GPR128 FGFR3-TACC3 FGFr3-BAIAP2L1 TMPRSS2-ERG/ETV1/ETV4 SLC45A3-FGFR2 ESRRA-C11orf20 PTPRK-RSPO3 EIF3E-RSPO2. Other fusion are contemplated, and non-fusion transcripts, proteins or other analytes suspected of colocalization are also in some cases selected for inclusion in separate probe sets.

Probe sets are generally discussed in the context of nucleic acid targets such as transcriptome constituent targets. However, non-RNA targets are also contemplated and suitable for probe based detection. DNA detection may be accomplished largely through the sample approaches used for RNA. Proteins and other analytes may be detected using aptamer probes having target identifying tags analogous to those discussed above. Alternately, proteins and other analytes may be detected using antibodies or other analyte binding moieties tagged with analyte identifying oligos. In other embodiments, proteins and other analytes may be detected using antibodies or other analyte binding moieties that may be analyte-tagged using secondary antibodies having analyte tags or other tags suitable for iterative code word assignment and detection.

Accordingly, combinations of analytes may be detected beyond transcript fusions. Transcripts which are not fused but are nonetheless co-localized may be detected through the disclosure herein. Protein-RNA complexes or protein complexes in particular loci on chromosomes, for example, are readily detected using a first probe set targeting proteins and a second probe set targeting nucleic acids. Similarly, protein complexes may be assayed for or detected de novo through the use of two sets of protein probes. In these cases, proteins suspected of complexing or co-occurring in common complexes may be probed for in separate probe sets.

Alternately, small molecule targets or binding partners may be detected through colocalization approaches herein. A first probe set may comprise, for example, aptamers that target the small molecule, while a second probe set may comprise probe sets targeting potential protein binding partners. Colocalization of the small molecule with one or more proteins, particularly at a resolution beyond the diffraction limint of the detection wavelength, may indicate that the small molecule targets or is bound by the colocalized protein.

Notably, due to the scale of targets that may be assayed in a single assay, the probe sets may be targeted or untargeted, such that binding partners may be detected even when a binding partner is unknown or incorrectly predicted prior to the assay. In addition, in the event that a small molecule or other analyte is known to be present in a sample and is nonetheless not detected by a probe set that is known to be able to bind to it, one may hypothesize that the small molecule or other analyte colocalizes with an analyte whose probe population is also present in the probe set concurrently applied, resulting in interference which precludes detection. By swapping the target analyte's probe population into a second probe set, one will temporally separate the binding and detection of the target analyte from the binding of probes to its unknown binding partner, thereby facilitating localization of both individually, and detection of their colocalization even if beyond the diffraction limit of the emission light used to detect them.

Accordingly, through some approaches herein, analyte-specific probe sets, that may anneal to or otherwise tag target analytes that are localized beyond (that is, closer than) the diffraction limit for the wavelength of the signal generated by the signal probe, are partitioned into separate populations such that they may be independently assayed, for example in separate temporally distinct or temporally interleaved assays of a sample. That is, a sample is contacted to a first population of analyte-specific probe sets so as to determine the location of a first population of target analytes, which are expected to be sufficiently separated from one another so as to facilitate iterative signal detection. The sample is contacted to a second population of analyte-specific probe sets, at least some sets of which are expected to bind target analytes which, if detected concurrently with the first population target analytes, would result in signals beyond (that is, closer than) the diffraction limit for detection, interfering with detection of both of these target analytes.

The first population and second population succeed in detecting their target analytes because their respective code words are not concurrently assayed for. In some cases, this is enabled by sample treatment which allows iterative sample assay events to be performed on a single sample. Alternately, this is enabled by sample treatment which allows interleaved sample assay events such that prolonged sample assaying does not destroy the sample.

In some cases the first population of analyte-specific probe sets is applied to the sample, and then decoding probes and signal probes are iteratively assed, so as to generate indirect, sequentially generated code words indicative of target analyte identity at a position in the sample. An image is taken and the iterative code words are deconvoluted or otherwise analyzed to determine target analyte position in the image.

The second population of analyte-specific probe sets is then applied to the sample, and second population decoding probes and signal probes are iteratively assed, so as to generate indirect, sequentially generated code words indicative of second population target analyte position in the sample. An image is taken and the iterative labels are deconvoluted or otherwise analyzed to determine target analyte position in the image.

In some cases, the signal probes of the first population of analyte-specific probe sets and the signal probes of the second population of analyte-specific probe sets share a common signal moiety, or emit at a common wavelength.

The images are then superimposed, by for example using in silico techniques, and target analyte signals which colocalize are indicative of target analytes that reside a distance outside of (closer than) the diffraction limit for the assay. Superimposition of images is in some cases facilitated or performed on a sample image taken before, concurrently with or subsequent to first probe set or second probe set data collection.

Alternative embodiments of these approaches differ in the timing of the iterative assaying of the first and second probe population analyte-specific probe sets. So long as the first population analyte specific probes and the second population analyte specific probes differ in their identifier elements such that decoding probes directed to the first population probes do not also bind to second population probes and such that decoding probes directed to the second population probes do not also bind to first population probes, then the first population and second population analyte specific probes do not need to be separately applied to a sample. Instead, in some embodiments both the first population and the second population analyte specific probes are concurrently applied to the sample, and the indirect sequential code words for analyte-specific probe sets are determined in a manner alternating from a first population signal to a second population signal and back until both the first population signal and the second population signal code words are determined in toto. So long as a first population signal and a second population signal are not concurrently captured, the alternating first and second population capture can be used to determine code words and target analyte position for both the first population analytes and the second population analytes.

Alternate embodiments use this image generation technology in combination with distinct data generation approaches, such as bead based or other sequencing. That is, making use of the relatively gentle impact of the iterative assays herein on sample integrity, one may perform am iterative tagging image generation on a sample, followed by a sequencing approach such as a bead-based positional sequencing approach. Such an approach allows one to obtain sequence information while obtaining positional information for transcripts or other nucleic acids within the diameter of the beads of one another, such as within 10 um of one another, down to within the resolution limit of the wavelength used to detect the analytes.

Yet other approaches may rely upon iterative data generation and sample preservation, but may not require the probe approach disclosed above. For example, according to the disclosure herein, bead or other sequence based positional sequencing approaches may be performed so as to accomplish resolution beyond the current limits of the technology, namely the microbead diameter in current microbead positional sequencing technologies. Through these approaches, a sample is subjected to successive rounds of bead-based library generation. So long as the beads contact points to a sample are offset relative to one another in successive rounds, target resolution may be achieved that is at the level of the overlap among bead positions in successive rounds rather than the diameter of the beads used in detection. These approaches require that at least two sets of position-tagged beads be generated for a given assay, and that that the at least two sets of position-tagged beads have nonidentical position tags, preferably such that they are offset relative to one another. The degree of offset may be governed by the number of rounds of bead binding to be applied to a sample. If two rounds of binding are to be performed, optimal offset is 50%, and resolution may be up to 50% of bead diameter. If three rounds of binding are to be performed, optimal offset is 33%, and resolution may be up to 33% of bead diameter. Similarly, for four rounds of binding, bead sets having 25$ offset may accomplish a resolution of 25% of the diameter of the beads. These improvements are substantial relative to the state of the art for transcript localization through bead-based positional sequencing, though they are substantially below the level of resolution achieved through iterative probe code word decoding as disclosed elsewhere herein.

Other, non-bead based in situ sequencing approaches are also known in the art, such as FISSEQ, disclosed in, for example, U.S. Pat. No. 11,447,807, published Sep. 20, 2022, which is hereby incorporated by reference in its entirety, and also US Publication No. 2021/0164039, published Jun. 3, 2021, which is hereby incorporated by reference in its entirety, and also U.S. Pat. No. 10,563,257, published Feb. 18, 2020, which is hereby incorporated by reference in its entirety. Other disclosures related to in situ sequencing are also known in the art.

Through in-situ sequencing approaches, resolution beyond the diffraction limit of the wavelength of the emission spectra is accomplished again through iterative detection events. That is, a first round of detection is performed, for which transcripts are detected up to a limit of the diffraction limit of the wavelength of the emission spectra. A second round of detection is performed, for which, again, transcripts are detected up to a limit of the diffraction limit of the wavelength of the emission spectra. However, superposition of the information in the first round and the second round allows colocalization of transcripts or other analytes beyond the diffraction limit when the superposition results in the signals overlapping. If obtained in a single round, the signals would likely interfere with one another, but when collected in temporally distinct (or interleaved) rounds, the diffraction limit may be overcome and the signals used to colocalize their corresponding transcripts or other analytes beyond the diffraction limit of the detection emission spectrum wavelength. In some cases the sequencing comprises targeted sequencing, particularly in the second round of sequencing, and may be informed by the identity of targets identified in the first round of sequencing. Thus, for example, if constituents of a fusion transcript are identified in a first round, the second suspected constituent of the putative fusion transcript may be assayed for in a targeted second sequencing round.

Due to the high specificity of target localization, one may perform large scale target detection on a sample. In some cases as many as 10, 20, 50, 100, 200, 500, 1,000, 2,000, 5,000, 10,000, 20,000 or more target analytes may be assayed for in a single sample section or in a single cell.

Furthermore, the methods and compositions disclosed herein are generally mild in their impact upon the sample material. This contrasts with some methods in the art, which comprise destruction of the sample pursuant to analyte localization. As a consequence, in many cases a sample may be imaged subsequent to or concurrently with or prior to analyte data collection, such that analyte localization data may be mapped onto an image of the sample from which the data was collected. Accordingly, disclosed herein are datasets and data images comprising analyte localization information, to a specificity beyond the diffraction limit of the electromagnetic wavelength used to detect them, superimposed upon an image of the sample from which the localization data was obtained.

Compositions and Kits Consistent with these Approaches

Accordingly, disclosed herein are compositions and kits for sequential signal-encoding approaches that facilitate detection of target analytes that are situated beyond (that is, within) a diffraction limit for detection. Some such compositions comprise a first population of analyte-specific probe sets for indirect sequential signal-encoding of target analytes in a sample, wherein probes of a first probe set target a first target analyte, and wherein no two analyte-specific probe sets are expected to bind to target analytes that have a separation less than a diffraction limit for a signal generated by a signal probe population comprising signal probes separately linked via decoding probes to the two analyte-specific probe sets. The number of analyte-specific probe sets in the population is often substantial, in some cases intended to cover a gene family, a panel of genes, proteins or transcripts or combinations thereof, up to and including a full cellular, tissue or organellar transcriptome or proteome. The first probe set population may in some cases comprise at least 100, 200, 500, 1,000, 2,000, 5,000, 10,000, 20,000, or more than 20,000 analyte-specific probe sets. These analyte-specific probe sets are selected such that their target analytes are not expected to be adjacent within a diffraction limit precluding their detection. Individual members of a probe set will, in contrast, target a common target analyte, but different analyte-specific probe sets are not expected or are selected to avoid or reduce the probability of identifying targets that are adjacent within a diffraction limit precluding their detection. In some cases these compositions or kits comprise a second population of analyte-specific probe sets, wherein at least one probe set of the second population binds a second population target analyte expected to have a separation less than a diffraction limit for a signal generated by a signal probe population. Examples of such target analytes include loci, transcript portions or chimeric protein portions that are known or suspected of being involved in chimeric translocations implicated in cancer. In some cases, the second population of analyte-specific probe sets comprises no more than 1/10 as many analyte-specific probe sets as the first population of analyte-specific probe sets, or no more than ⅕ as many analyte-specific probe sets as the first population of analyte-specific probe sets.

The populations are in some cases distinguished by having no-overlapping sets of identifier elements (T), such that identifier elements in the first population are not found in the second population.

In an embodiment of the disclosure the sample is a biological sample, preferably comprising biological tissue, further preferably comprising biological cells. A biological sample may be derived from an organ, organoids, cell cultures, stem cells, cell suspensions, primary cells, samples infected by viruses, bacteria or fungi, eukaryotic or prokaryotic samples, smears, disease samples, a tissue section.

The method is particularly qualified to encode, identify, detect, count or quantify analytes or single analytes molecules in a biological sample, such as a sample which contains nucleic acids or proteins as said analytes. It is understood that the biological sample may be in a form as it is in its natural environment (for example, liquid, semi-liquid, solid etc.), or processed, e.g. as a dried film on the surface of a device which may be re-liquefied before the method is carried out.

In another embodiment of the disclosure prior to step (2) the biological tissue and/or biological cells are fixed. For example, in some embodiments, the cell and/or the tissue is fixed prior to introducing the probes, e.g., to preserve the positions of the analytes like nucleic acids within the cell. Techniques for fixing cells are known to those of ordinary skill in the art. As non-limiting examples, a cell may be fixed using chemicals such as formaldehyde, paraformaldehyde, glutaraldehyde, ethanol, methanol, acetone, acetic acid, or the like. In one embodiment, a cell may be fixed using Hepes-glutamic acid buffer-mediated organic solvent (HOPE).

This measure has the advantage that the analytes to be encoded, e.g. the nuclei acids or proteins, are immobilized and cannot escape. In doing so, the analytes then prepared for a better detection or encoding by the method according to the disclosure.

In yet a further embodiment within the set of analyte-specific probes the individual analyte-specific probes comprise binding elements (S1, S2, S3, S4, S5) which specifically interact with different sub-structures of one of the analytes to be encoded.

By this measure the method becomes even more robust and reliable because the signal intensity obtained at the end of the method or a cycle, respectively, is increased. It is understood, that the individual probes of a set while binding to the same analyte differ in their binding position or binding site at or on the analyte. The binding elements S1, S2, S3, S4, S5 etc. of the first, second, third fourth, fifth etc. analyte-specific probes therefore bind to or at a different position which, however, may or may not overlap.

In an advantageous embodiment, the present disclosure pertains to kit for multiplex analyte encoding, comprising:
(A1) at least a first set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
and
(A2) at least a second set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T), and
wherein the analyte-specific probes in each set of analyte-specific probes binds to the same analyte and comprises the same nucleotide sequence of the identifier element (T) which is unique to said analyte; and
wherein (optionally) the number of probes and/or targets of first set of analyte-specific probes according to step A1 (the transcript plexity of A1) is at least 10 times higher than the number of probes and/or targets of the second set of analyte-specific probes according to step A2 (the transcript plexity of A2); and
(B1) at least a first set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide; and
(B2) at least a second set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(C) a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.

A multiplex method or assay allows the simultaneous measurement of multiple analytes according to the present disclosure. It may be used to determine the presence or absence of a plurality of predetermined (known) analytes like nucleic acid target sequences in a sample. An analyte may be “predetermined” in that its sequence is known to design a probe that binds to the that target.

In some advantageous embodiments according to the present disclosure at least 2, at least 5, at least 10, at least 15, at least 20, in particular at least 25, in particular at least 30 different analytes are detected and/or quantified in a sample. For example, there may be at least 5, at least 10, at least 20, at least 50, at least 75, at least 100, at least 300, at least 1,000, at least 3,000, at least 10,000, or at least 30,000 distinguishable analyte-specific probes that are applied to a sample, e.g., simultaneously or sequentially.

In the multiplexing methods of the present disclosure, in particular at least 2 different subgroups of analytes (e.g., mRNA molecules) that is, tags with spatially overlap (a distance beyond, exceeding or below the diffraction limit of the respective microscope) are targeted. Thus, the present invention allows an increased resolution and higher “information density” as compared to conventional methods.

With higher “information density” the number of diagnostic conclusions is meant, which can be drawn from a single sample, thereby reducing the number of samples needed and the time needed for a specific result. It also decreases the workload of the personal performing the analysis and/or allows a higher throughput of samples as compared to conventional methods.

In some advantageous embodiments, at least 4 rounds to collect information for identification of the analyte are carried out, wherein multiple readout increases the accuracy of identification and avoids false positives. The unique tag can be identified by various techniques, including hybridization, e.g., with labeled probes, directly or indirectly or by sequencing (by synthesis, ligation). In particular, the identity of the tag can be encoded with one single signal (binary code), two or more signals, wherein the signal can be a fluorescent label (e.g., attached to an oligonucleotide).

In some advantageous embodiments according to the present disclosure, the kit does not comprise sets of analyte-specific probes as defined under item A1) and A2).

Preferably, if the analyte in the kits or methods according to the present disclosure is a nucleic acid, each set of analyte-specific probes comprises at least five (10) analyte-specific probes, in particular at least fifteen (15) analyte-specific probes, in particular at least twenty (20) analyte-specific probes which specifically interact with different sub-structures of the same analyte. Nucleic acid analyte includes specific DNA molecules, e.g. genomic DNA, nuclear DNA, mitochondrial DNA, viral DNA, bacterial DNA, extra- or intracellular DNA etc., and specific mRNA molecules, e.g. hnRNA, miRNA, viral RNA, bacterial RNA, extra- or intracellular RNA, etc.

Preferably, if the analyte in the kits or methods according to the present disclosure is a peptide, a polypeptide or a protein, each set of analyte-specific probes comprises at least two (2) analyte-specific probes, in particular at least three (3) analyte-specific probes, in particular at least four (4) analyte-specific probes which specifically interact with different sub-structures of the same analyte.

In some advantageous embodiments according to the present disclosure the kit comprises at least two different sets of signal oligonucleotides, wherein the signal oligonucleotides in each set comprise a different signal element and comprise a different connector element (C).

In particular, the kit may comprise at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets per analyte differ in the translator element (c) comprising a nucleotide sequence allowing a specific hybridization of a signal oligonucleotide.

In some embodiments the kit comprises at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets for at least one analyte differ in the translator element (c) comprising a nucleotide sequence allowing a specific hybridization of a signal oligonucleotide.

In some advantageous embodiments, the number of different sets of decoding oligonucleotides per analyte comprising different translator elements (c) corresponds to the number of different sets of signal oligonucleotides comprising different connector elements (C). However, the decoding oligonucleotides in a particular set of decoding oligonucleotides may interact with identical identifier elements (T) which are unique to a particular analyte. In particular, all sets of decoding oligonucleotides for the different analytes may comprise the same type(s) of translator element(s) (c). In another aspect, the present disclosure is generally directed to methods including acts of exposing a sample to a plurality of analyte-specific probes; for each of the analyte-specific probes, determining binding of the analyte-specific probes within the sample; creating codewords based on the binding of the analyte-specific probes, the decoding oligonucleotides and the signal oligonucleotides; and for at least some of the codewords, matching the codeword to a valid codeword. In certain embodiments, this pattern of binding or hybridization of the analyte-specific probes, the decoding oligonucleotides and the signal oligonucleotides may be converted into a “codeword.” For example, for instance, the codewords may be “101” and “110” for a first analyte and a second analyte, respectively, where a value of 1 represents binding and a value of 0 represents no binding of decoding oligonucleotides and/or the binding of signal oligonucleotides without and/or quenched signal element. The analyte in the detection round/cycle is therefore not detectable during imaging.

To create such a zero (0) in a codeword for an individual analyte the kit may comprise:
(D) at least a set of non-signal decoding oligonucleotides for binding to a particular identifier element (T) of analyte-specific probes, wherein the decoding oligonucleotides in the same set of non-signal decoding oligonucleotides interacting with the same different identifier element (T),
wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

To create such a zero (0) in a codeword for an individual analyte the kit may comprise:
(D) at least a set of non-signal decoding oligonucleotides for binding to a particular identifier element
(T) of analyte-specific probes, wherein the decoding oligonucleotides in the same set of non-signal decoding oligonucleotides interacting with the same different identifier element (T),
wherein each non-signal decoding oligonucleotide comprises an identifier connector element (t) comprising a nucleotide sequence which is essentially complementary to at least a section of a unique identifier sequence, and comprises a translator element that does not interact/bind to a signal oligonucleotide due to an instable binding sequence and/or due to the translator element is to short (c) comprising a nucleotide sequence allowing a specific hybridization of a signal oligonucleotide.

In some advantageous embodiments, the kit comprises:
(D) at least two (2) different sets of non-signal decoding oligonucleotides for binding to at least two different identifier elements (T) of analyte-specific probes, each set of non-signal decoding oligonucleotides interacting with a different identifier element (T),
wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

In some advantageous embodiments, the different sets of non-signal decoding oligonucleotides may be comprised in a pre-mixture of different sets of non-signal decoding oligonucleotides or exist separately.

Furthermore, in some advantageous embodiments the kit may comprises:
(E) a set of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

In some advantageous embodiments, the kit comprises:
(E) at least two sets of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

In some advantageous embodiments, the different sets of non-signal oligonucleotides may be comprised in a pre-mixture of different sets of non-signal oligonucleotides or exist separately.

Further, in some embodiments the decoding oligonucleotides in a particular set of decoding oligonucleotides interacts with identical identifier elements (T) which are unique to a particular analyte.

In some advantageous embodiments, the different sets of decoding oligonucleotides may be comprised in a pre-mixture of different sets of decoding oligonucleotides or exist separately. In some advantageous embodiments, the different sets of analyte-specific probes may be comprised in a pre-mixture of different sets of analyte-specific probes or exist separately. In some advantageous embodiments, the different sets of signal oligonucleotides may be comprised in a pre-mixture of different sets of signal oligonucleotides or exist separately.

In some advantageous embodiments, a mixture of decoding oligonucleotides and/or multi-decoders is provided that specifically hybridize to the unique identifier sequences of the probe sets. In some embodiments, the decoding oligonucleotides comprise of at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide. In some embodiments multi-decoders comprising at least three sequence elements are used, a first element that is complementary to the unique identifier sequences of the corresponding probe set and at least to additional sequence elements (translator elements) that provide sequences for the specific hybridization of at least two different signal oligonucleotides. The translator elements define the type of signals that are recruited to the multi-decoder. Different possible structures of a multi-decoder can be seen in FIG. 15. Since a multi-decoder does recruit a full signal oligonucleotide per translator element, the brightness of the signals in each channel is not lower than the brightness of signals with decoding oligonucleotides.

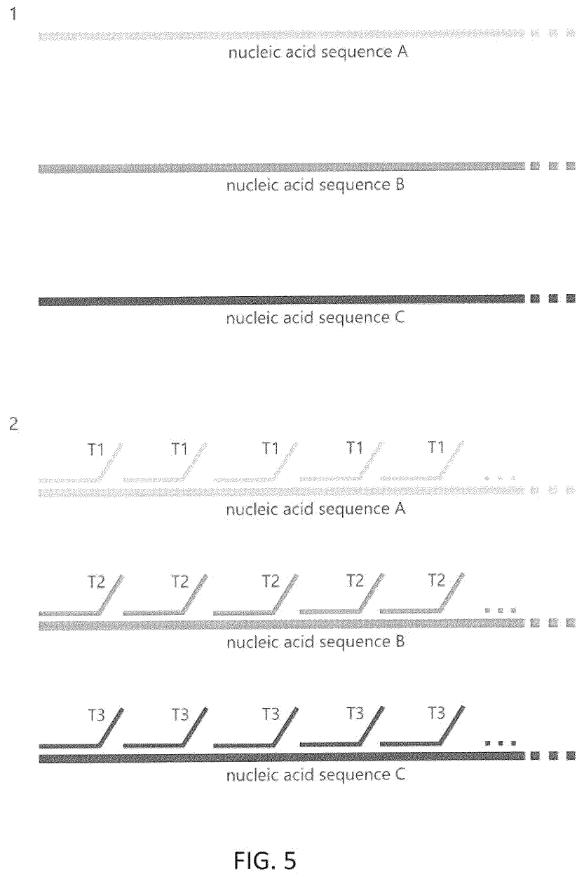

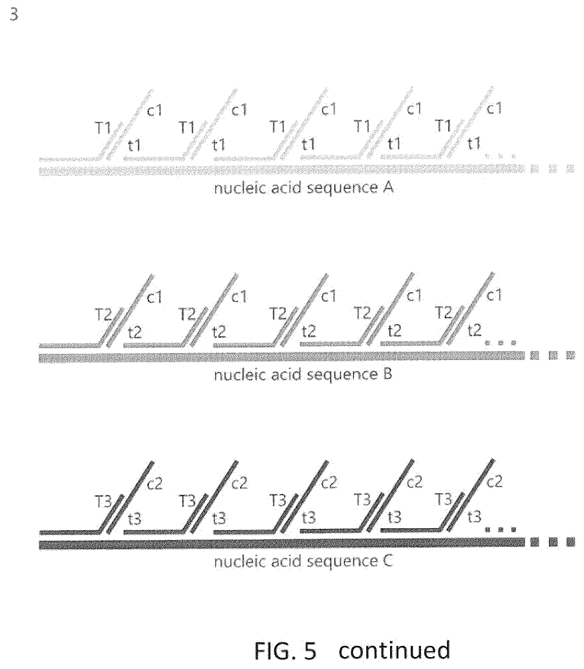

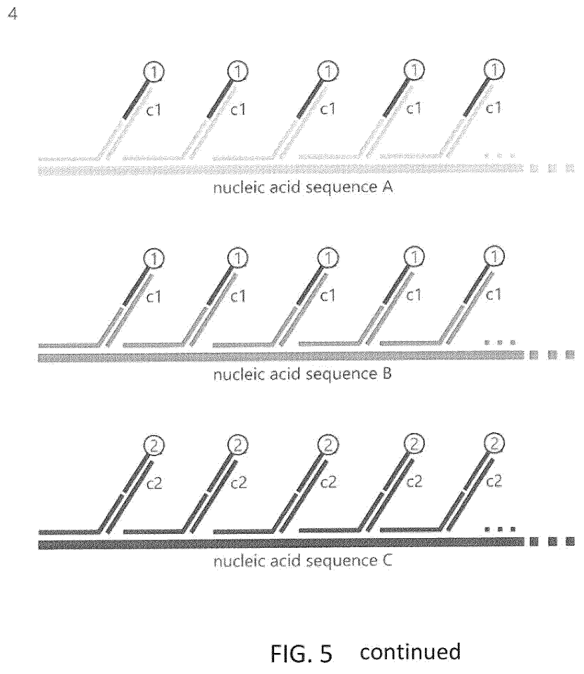

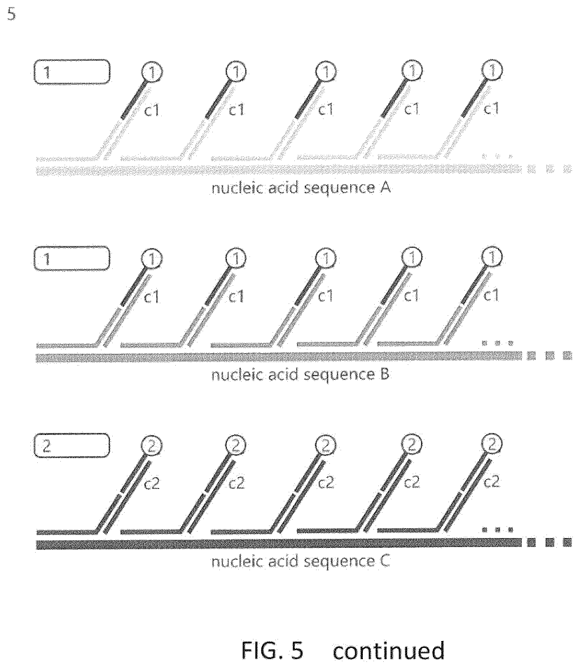

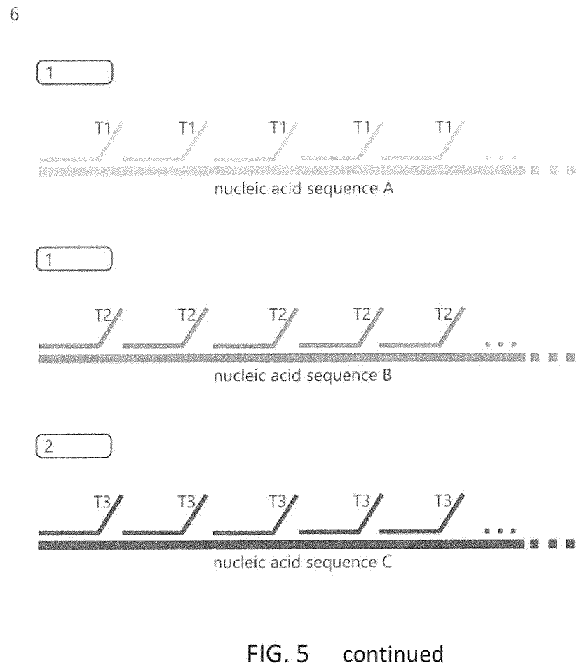

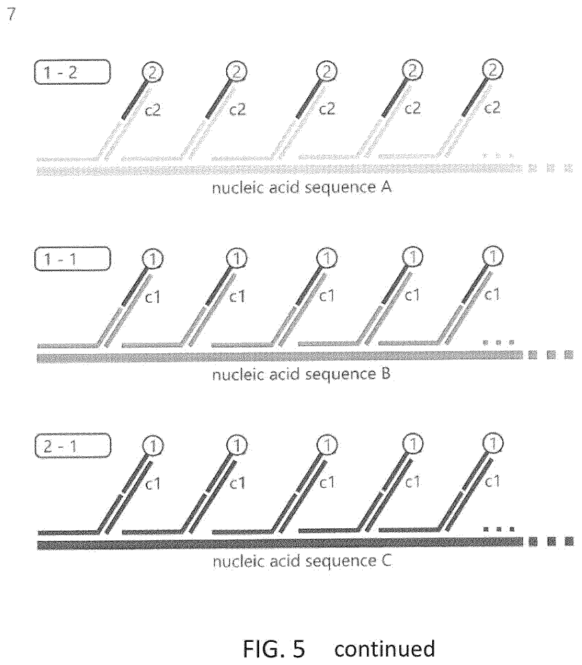

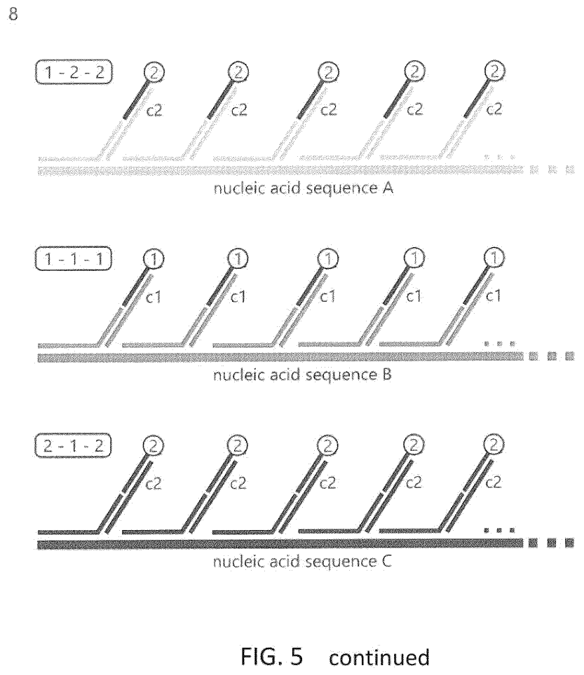

The usage of multi-decoders increases further the efficiency of the encoding scheme. FIG. 16 shows a possible encoding scheme using multi-decoders based upon the same conditions used for the examples with the decoding oligonucleotide with two sequence elements. One can clearly see that the multi-decoder-based encoding scheme can create a higher hamming distance, with the same number of rounds and the same number of different signal oligonucleotides used in the example of FIG. 5.

As mentioned above the analyte to be encoded may be a nucleic acid, preferably DNA, PNA or RNA, in particular mRNA, a peptide, polypeptide, a protein, and/or mixtures thereof.

In some advantageous embodiments, the binding element(S) comprises an amino acid sequence allowing a specific binding to the analyte to be encoded. The binding element(S) may comprise moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof.

In some advantageous embodiments, the binding element(S) may comprise or is an antibody or an antibody fragment selected from the group consisting of Fab, scFv; single domain, or a fragment thereof, bis scFv, F (ab) 2, F (ab) 3, minibody, diabody, triabody, tetrabody and tandab.

The present disclosure pertains in particular to a multiplex method for detecting different analytes in a sample by sequential signal-encoding of said analytes, comprising the steps of:
(A1) contacting the sample with at least a first set of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(A2) contacting the sample with at least a second set of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
   wherein (optionally) the number of probes and/or targets of first set of analyte-specific probes according to step A1 (the transcript plexity of A1) is at least 10 times higher than the number of probes and/or targets of the second set of analyte-specific probes according to step A2 (the transcript plexity of A2); and
(B1) contacting the sample with at least a first set of decoding oligonucleotides per analyte set according to A1, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(B2) contacting the sample with at least a second set of decoding oligonucleotides per analyte set according to A2, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(C) contacting the sample with at least a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.
(D) Detecting the signal caused by the signal element;
(E) selectively removing the decoding oligonucleotides and signal oligonucleotides from the sample, thereby essentially maintaining the specific binding of the analyte-specific probes to the analytes to be encoded;
(F) Performing at least three (3) further cycles comprising steps B) to E) to generate an encoding scheme with a code word per analyte, wherein in particular the last cycle may stop with step (D).

As mentioned above, the method according to the present disclosure comprises selectively removing the decoding oligonucleotides and signal oligonucleotides from the sample, thereby essentially maintaining the specific binding of the analyte-specific probes to the analyte to be encoded. In particular all steps are performed sequentially. However, some steps may be performed simultaneously, in particular the contacting steps A) to C), in particular B) and C).

By this measure the requirements for another round/cycle of binding further decoding oligonucleotides to the same analyte-specific probes are established, thus finally resulting in a code or encoding scheme comprising more than one signal. This step is realized by applying conditions and factors well known to the skilled person, e.g., pH, temperature, salt conditions, oligonucleotide concentration, polymers etc.

In another embodiment of the present disclosure, the method may comprise repeating steps (B)-(E) at least three times to generate an encoding scheme. With this measure a code of four signals in case of four cycles/rounds which are carried out by the user, where ‘n’ is an integer representing the number of rounds. The encoding capacity of the method according to the disclosure is herewith increased depending on the nature of the analyte and the needs of the operator. In an embodiment of the disclosure said encoding scheme is predetermined and allocated to the analyte to be encoded.

However, this measure enables a precise experimental set-up by providing the appropriate sequential order of the employed decoding and signal oligonucleotides and, therefore, allows the correct allocation of a specific analyte to a respective encoding scheme. The decoding oligonucleotides which are used in repeated steps (B)-(D2) may comprise a translator element (c2) which is identical with the translator element (c1) of the decoding oligonucleotides used in previous steps (B)-(E). In another embodiment of the disclosure decoding oligonucleotides are used in repeated steps (B)-(E) comprising a translator element (c2) which differs from the translator element (c1) of the decoding oligonucleotides used in previous steps (B)-(E). It is understood that the decoding elements may or may not be changed from round to round, such as in the second round (B)-(E) comprising the translator element c2, in the third round (B)-(E) comprising the translator element c3, in the fourth round (B)-(E) comprising the translator element c4 etc., wherein ‘n’ is an integer representing the number of rounds.

The signal oligonucleotides which are used in repeated steps (B)-(E) may comprise a signal element which is identical with the signal element of the decoding oligonucleotides used in previous steps (B)-(E). In a further embodiment of the disclosure signal oligonucleotides are used in repeated steps (B)-(E) comprising a signal element which differs from the signal element of the decoding oligonucleotides used in previous steps (B)-(E). In some embodiments no-signal oligonucleotides and/or no-signal decoding oligonucleotides for an individual analyte are used, resulting to the value 0 in the codeword for this cycle/position. In some embodiments in a repeated cycle no decoding oligonucleotides for an individual analyte is contacted with the sample resulting also to the value 0 in the codeword for this cycle/position.

By this measure each round the same or a different signal is provided resulting in an encoding scheme characterized by a signal sequence consisting of numerous different signals. This measure allows the creation of a unique code or code word which differs from all other code words of the encoding scheme. In another embodiment of the disclosure, the binding element(S) of the analyte-specific probe comprises a nucleic acid comprising a nucleotide sequence allowing a specific binding to the analyte to be encoded, preferably a specific hybridization to the analyte to be encoded.

In some advantageous embodiments, all steps are automated, in particular wherein steps B) to F) are automated, in particular by using a robotic system and/or an optical multiplexing system according to the present disclosure. In some examples, the steps may be performed in a fluidic system.

As mentioned above, with the methods according to the present disclosure an encoding scheme with a code word per analyte set is generated. Therefore, each analyte set may be associated with a specific code word, wherein said code word comprise several positions, and wherein each position corresponds to one cycle resulting in a plurality of distinguishable encoding schemes with the plurality of code words. In particular, said encoding scheme may be predetermined and allocated to the analyte to be encoded.

In some advantageous embodiments, the code words obtained for the individual analytes in the performed cycles comprise the detected signals and additionally at least one element corresponding to no detected signal like 0,1 or 0,1,2 etc. (see also FIG. 13 and FIG. 14). In particular, no signal is detected for at least one analyte within at least one cycle if using a non-signal probe according to FIG. 14, Nr. 2 to 4, or a non-signal decoding oligonucleotide as shown in FIG. 14, Nr. 5, or if in one cycle no decoding oligonucleotide is contacted with the corresponding identifier sequence comprised on analyte-specific probe interacting with the corresponding analyte in the sample. In this cycle the position has the value zero (0).

In some advantageous embodiments, at least for one individual analyte a position of the code word is zero (0). In particular, the code word zero (0) is generated by using no decoding oligonucleotides having an identifier connector element (t) comprising a nucleotide sequence which is essentially complementary to at least a section of the unique identifier sequence of the identifier element (T) of a corresponding analyte-specific probe for an individual analyte. As mentioned above, in some embodiments, if at least for one individual analyte a position of the code word is zero (0) in this cycle no corresponding decoding oligonucleotides having an identifier connector element (t) comprising a nucleotide sequence which is essentially complementary to at least a section of the unique identifier sequence of the identifier element (T) of a corresponding analyte-specific probe for an individual analyte are used.

Furthermore, in some advantageous embodiments, the sample is contacted with at least two different sets of signal oligonucleotides, wherein the signal oligonucleotides in each set comprise a different signal element and comprise a different connector element (C).

In more particular embodiments, the sample is contacted with at least two different sets of decoding oligonucleotides per analyte,
wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and
wherein the decoding oligonucleotides of the different sets per analyte differ in the translator element (c) comprising a nucleotide sequence allowing a specific hybridization of a signal oligonucleotide.

In more particular embodiments, the sample is contacted with at least two different sets of decoding oligonucleotides per analyte,
wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and
wherein the decoding oligonucleotides of the different sets per analyte differ in the translator element (c) comprising a nucleotide sequence allowing a specific hybridization of a signal oligonucleotide;
wherein only one set of decoding oligonucleotides per analyte is used per cycle, and/or wherein different sets of decoding oligonucleotides are used in different cycles in combination with the corresponding set of signal oligonucleotides in the same cycle.

In some advantageous embodiments, the number of different sets of decoding oligonucleotides per analyte comprising different translator elements (c) corresponds to the number of different sets of signal oligonucleotides comprising different connector elements (C). All sets of decoding oligonucleotides for the different analytes may comprise the same type(s) of translator element(s) (c). In another aspect the translator element(s) (c) may differ between sets.

In some advantageous embodiments of the method according to the present disclosure, the sample is contacted with at least a set of non-signal decoding oligonucleotides for binding to a particular identifier element (T) of analyte-specific probes, wherein the decoding oligonucleotides in the same set of non-signal decoding oligonucleotides interacting with the same different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

As mentioned above, the sample may be contacted with at least two (2) different sets of non-signal decoding oligonucleotides for binding to at least two different identifier elements (T) of analyte-specific probes, each set of non-signal decoding oligonucleotides interacting with a different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

In some advantageous embodiments of the method according to the present disclosure, the different sets of non-signal decoding oligonucleotides may be comprised in a pre-mixture of different sets of non-signal decoding oligonucleotides or exist separately.

Furthermore, in some advantageous embodiments of the method according to the present disclosure, the sample is contacted with a set of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

In further embodiments, the sample may be contacted with:
at least two sets of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

As mentioned above, the different sets of non-signal oligonucleotides may be comprised in a pre-mixture of different sets of non-signal oligonucleotides or exist separately.

In further embodiments, the decoding oligonucleotides in a particular set of decoding oligonucleotides interacts with identical identifier elements (T) which are unique to a particular analyte.

As mentioned above, the different sets of decoding oligonucleotides may be comprised in a pre-mixture of different sets of decoding oligonucleotides or exist separately as well as the different sets of analyte-specific probes may be comprised in a pre-mixture of different sets of analyte-specific probes or exist separately as well the different sets of signal oligonucleotides may be comprised in a pre-mixture of different sets of signal oligonucleotides or exist separately.

In some advantageous embodiments of the method according to the present disclosure, the binding element(S) comprise a nucleic acid comprising a nucleotide sequence allowing a specific binding to the analyte to be encoded, preferably a specific hybridization to the analyte to be encoded.

In some advantageous embodiments of the method according to the present disclosure, after step A) and before step B) the non-bound analyte-specific probes may be removed, in particular by washing, further after step B) and before step C) the non-bound decoding oligonucleotides may be removed, in particular by washing further, after step C) and before step D) the non-bound signal oligonucleotides may be removed, in particular by washing.

In some advantageous embodiments of the method according to the present disclosure, the analyte specific probes may be incubated with the sample, thereby allowing a specific binding of the analyte specific probes to the analytes to be encoded, further the decoding oligonucleotides may be incubated with the sample, thereby allowing a specific hybridization of the decoding oligonucleotides to identifier elements (T) of the respective analyte-specific probes, further the signal oligonucleotides may be incubated with the sample, thereby allowing a specific hybridization of the signal oligonucleotides to translator elements (T) of the respective decoding oligonucleotides.

As mentioned above, the analyte to be encoded may be a nucleic acid, preferably DNA, PNA, RNA, in particular mRNA, a peptide, polypeptide, a protein or combinations thereof. Therefore, the binding element(S) may comprise an amino acid sequence allowing a specific binding to the analyte to be encoded. Examples for a binding element(S) are moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof. In particular, the binding element(S) is an antibody, or an antibody fragment selected from the group consisting of Fab, scFv; single domain, or a fragment thereof, bis scFv, Fab 2, Fab 3, minibody, diabody, triabody, tetrabody and tandab.

By this measure the method is further developed to such an extent that the encoded analytes can be detected by any means which is adapted to visualize the signal element. Examples of detectable physical features include e.g., light, chemical reactions, molecular mass, radioactivity, etc.

In some advantageous embodiments, the signal caused by the signal element, therefore in particular the binding of the signal oligonucleotides to the decoding oligonucleotides, interacting with the corresponding analyte probes, bound to the respective analyte is determined by:
(a) Imaging at least a portion of the sample; and/or
(b) Using an optical imaging technique; and/or
(c) Using a fluorescence imaging technique; and/or
(d) Multi-color fluorescence imaging technique; and/or
(e) Super-resolution fluorescence imaging technique.

Since the signals are encoded in certain colors, in some aspects color filters may be employed to differentiate the signals of each color vs. the total signal. Thereby facilitating additional information.

The kits and method according to the present disclosure may be used ideally for in vitro methods for diagnosis of a disease, such as a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases.

Further, the kits and method according to the present disclosure may be used also ideally for in vitro methods for diagnosis of a disease in plants, such as a disease selected from the group comprising: diseases caused by biotic stress, preferably by infectious and/or parasitic origin, or diseases caused by abiotic stress, preferably caused by nutritional deficiencies and/or unfavorable environment.

Further, the kits and method according to the present disclosure may be used also ideally for in vitro methods for screening, identifying and/or testing a substance and/or drug comprising:
(a) contacting a test sample comprising a sample with a substance and/or drug
(b) detecting different analytes in a sample by sequential signal-encoding of said analytes with a method according to the present disclosure.

An optical multiplexing system suitable for the method according to the present disclosure, comprising at least:
a reaction vessel for containing the kits or part of the kits according to the present disclosure;
a detection unit comprising a microscope, in particular a fluorescence microscope;
a camera;
a liquid handling device.

In some embodiments, optical multiplexing system may comprise further a heat and cooling device and/or a robotic system.

In some embodiments, the method according to the present disclosure encodes a nucleic acid analyte, such as an mRNA, e.g., such an mRNA coding for a particular protein.

In some advantageous embodiments, the method described herein is used for specific detection of many different analytes. The technology allows to distinguish a higher number of analytes than different signals are available. The process includes at least four consecutive rounds of specific binding, signal detection and selective denaturation (if a next round is required), eventually producing a signal code. To decouple the dependency between the analyte specific binding and the oligonucleotides providing the detectable signal, a so called “decoding”-oligonucleotide is introduced. The decoding oligonucleotide transcribes the information of the analyte specific probe set to the signal oligonucleotides.

In a specific embodiment the method may comprise the steps of 1. providing one or more analyte specific probe sets, the set of analyte specific probes consist of one or more different probes, each differing in the binding moiety that specifically interacts with the analyte, all probes of a single probe set are tethered to a sequence element (unique identifier), that is unique to a single probe set and allows the specific hybridization of a decoding oligonucleotide, 2. specific binding of the probe sets to their target binding sites of the analyte, 3. eliminating non-bound probes (e.g. by a wash step), 4. providing a mixture of decoding oligonucleotides that specifically hybridize to the unique identifier sequences of the probe sets, the decoding oligonucleotides comprise of at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide, 5. specific hybridization of the decoding oligonucleotides to the unique identifier sequences provided by the bound probe sets, 6. eliminating non-bound decoding oligonucleotides (e.g. by washing step), 7. providing a mixture of signal oligonucleotides, comprising of a signal that can be detected and a nucleic acid sequence that specifically hybridizes to the translator element of one of the decoding oligonucleotides used in the former hybridization step, 8. specific hybridization of the signal oligonucleotides, 9. eliminating non-bound signal oligonucleotides, 10. detection of the signals, 11. selective release of decoding oligonucleotides and signal oligonucleotides while the binding of specific probe sets to the analyte is almost or completely unaffected, 12. eliminating released decoding oligonucleotide and signal oligonucleotides (e.g. by a washing step) while the binding of specific probes sets to the analytes is almost or completely unaffected, repeating the steps 4 to 12 at least three times until the detection of a sufficient number of signals to generate an encoding scheme for each different analyte of interest.

It is to be understood that the before-mentioned features and those to be mentioned in the following cannot only be used in the combination indicated in the respective case, but also in other combinations or in an isolated manner without departing from the scope of the disclosure.

The disclosure is now further explained by means of embodiments resulting in additional features, characteristics and advantages of the disclosure. The embodiments are of pure illustrative nature and do not limit the scope or range of the disclosure. The features mentioned in the specific embodiments are general features of the disclosure which are not only applicable in the specific embodiment but also in an isolated manner in the context of any embodiment of the disclosure.

The method disclosed herein is used for specific detection of many different analytes. The technology allows distinguishing a higher number of analytes than different signals are available. The process preferably includes at least two consecutive rounds of specific binding, signal detection and selective denaturation (if a next round is required), eventually producing a signal code. To decouple the dependency between the analyte specific binding and the oligonucleotides providing the detectable signal, a so called “decoding” oligonucleotide is introduced. The decoding oligonucleotide transcribes the information of the respective analyte specific probe set to the signal oligonucleotides.

The present disclosure pertains further to methods of detecting an analyte, comprising: attaching a plurality of analyte-specific probes to the analyte, wherein the analyte-specific probes independently attach to the analyte and wherein the analyte-specific probes share a common identifier segment (T); annealing a plurality of first decoding oligonucleotides to the analyte-specific probes, wherein the first decoding oligonucleotides share a first common region that is reverse complementary to the common identifier segment and a second common region; annealing a first signal oligonucleotide to at least one of the plurality of first decoding oligonucleotides such that an oligo tethered to the first signal oligonucleotide is reverse complementary to the second common region; detecting the first signal oligonucleotide; removing the plurality of first decoding oligonucleotides; annealing a plurality of second decoding oligonucleotides to the analyte-specific probes, wherein the second decoding oligonucleotides share a first common region that is reverse complementary to the common identifier segment and a second decoding oligonucleotides second common region that differs from the second common region of the first decoding; annealing a second signal oligonucleotide to at least one of the plurality of second decoding oligonucleotides such that an oligo tethered to the signal oligonucleotide is reverse complementary to the second decoding oligonucleotide second common region; and detecting the second signal oligonucleotide.

In particular, in the above mentioned embodiment a second aliquot of a plurality of first decoding oligonucleotides is annealed to the analyte-specific probes. Furthermore, a first aliquot of a plurality of first decoding oligonucleotides is annealed to the analyte-specific probes.

In some further advantageous embodiments, the present disclosure pertains to a method of assigning an analyte to a position in an image, comprising assigning a fluorescence pattern to the analyte, observing the fluorescence pattern at the position in the image, and assigning the analyte to the position, in particular wherein observing the fluorescence pattern comprises repeating steps of labeling the position using a fluorophore tagged oligo drawn from a re-accessible pool, performing a single excitation at the position in the image, and contacting the analyte to a denaturant, in particular wherein observing the fluorescence pattern comprises repeating steps of labeling the position using a fluorophore tag-recruiting bridging oligo drawn from a re-accessible pool, performing a single excitation at the position in the image, and contacting the analyte to a denaturant.

In some further advantageous embodiments, the present disclosure pertains to a composition comprising a cell having nucleic acids distributed therein, wherein a first nucleic acid is tagged by a first plurality of probes that target adjacent segments of the first nucleic acid and that share a common first tether segment; a second nucleic acid is tagged by a second plurality of probes that target adjacent segments of the second nucleic acid and that share a common second tether segment; and a third nucleic acid is tagged by a third plurality of probes that target adjacent segments of the third nucleic acid and that share a common third tether segment; a first adapter population comprising molecules having a first tether reverse complementary region and a first fluorophore adapter tether; a second adapter population comprising molecules having a second tether reverse complementary region and a second fluorophore adapter tether; a third adapter population comprising molecules having a third tether reverse complementary region and a first fluorophore adapter tether; a population of first fluorophores having a first tether reverse complementary region; and a population of second fluorophores having a second tether reverse complementary region.

In some further advantageous embodiments, the present disclosure pertains to a method of assigning coded fluorescence patterns to a plurality of target analytes in a cell, comprising: subjecting the cell to a plurality of detection rounds, each detection round comprising: contacting the cell to representatives of the same at least two populations of tagged fluorescence moieties, and removing the fluorescent moieties after a single excitation event, in particular wherein the number of patterns detectable increases exponentially with the number of detection rounds, wherein the fluorescence moieties are not tagged with nucleic acid tags that are specific to the target nucleic acids, and wherein separate aliquots of common tagged fluorescence moieties are used across multiple detection rounds.

In particular, with the above notified method a total decoding efficiency of at least 20%, at least 30%, at least 40%, at least 50%, at least 60% may be achieved. In some aspects the total decoding efficiency is 100% or less, 99% or less, 98.5% or less, 90% or less, 80% or less. In some aspects the total decoding efficiency between 20% and 100%, between 30% and 90%, between 40% and 80%.

In some further advantageous embodiments, the present disclosure pertains to a method of assigning coded fluorescence patterns to a plurality of target analytes in a cell, comprising: contacting a target to a bipartite labeling probe, the bipartite labeling probe comprising a target-specific moiety and a fluorophore-specifying moiety; contacting the bipartite labeling probe to a first aliquot of a fluorophore reservoir comprising no more than two populations of fluorophores; replacing the fluorophore specifying moiety in the bipartite probe, and contacting the bipartite labeling probe to a second aliquot of the fluorophore reservoir comprising the same no more than two populations.

In some embodiments of the above notified method, replacing the fluorophore specifying moiety in the bipartite probe comprises denaturing a binding between a target-specific moiety and a fluorophore-specifying moiety after subjecting the bipartite labeling probe bound to a fluorophore of the fluorophore to excitation energy. In particular, replacing the fluorophore specifying moiety in the bipartite probe comprises drawing from one of no more than two fluorophore specifying moiety reservoirs.

In some further advantageous embodiments, the present disclosure pertains to a method of detecting an analyte, comprising: attaching a plurality of probes to the analyte, in particular a nucleic acid, wherein the probes independently attach/anneal to the analyte and wherein the probes share a common identifier segment; annealing a plurality of first adapter segments to the probes, wherein the first adapter segments share a first common region that is reverse complementary to the common identifier segment and a second common region, in particular configured to accommodate a single reporter/selected from no more than two reporter categories; annealing a first reporter to at least one of the plurality of first adapter segments such that an oligo tethered to the first reporter is reverse complementary to the second common region; detecting the first reporter; removing the plurality of first adapter segments, in particular without annealing a second reporter to the at least one of the plurality of first adapter segments; annealing a plurality of second adapter segments to the probes, wherein the second adapter segments share a first common region that is reverse complementary to the common identifier segment and a second adapter second common region that differs from the second common region of the first adapter segments, in particular configured to accommodate a single reporter/selected from no more than two reporter categories; annealing a second reporter to at least one of the plurality of second adapter segments such that an oligo tethered to the second reporter is reverse complementary to the second adapter second common region; and detecting the second reporter, in particular without annealing a third reporter to the at least one of the plurality of first adapter segments.

### C. Quality Control Approaches

Also disclosed herein are quality control approaches to detect signals which arise from distinct analyte-specific probe sets that have bound targets situated beyond (that is, within) a diffraction limit for detection. Some of these approaches rely upon the adoption of iterative label ‘words’ for a given analyte that comprise both emission and non-emission or blank signal to form a target specific iterative label. In particular, use of unlabelled signal oligonucleotides in an iterative label allows one to build an iterative label for a target analyte comprising both signal and lack of signal at various positions in the iterative label. If adjacent targets are situated beyond (that is, within) a diffraction limit for detection, then their iterative labels will overlap and at each position in the iterative label detected, one will receive signal from not one but both of the iterative label signal oligonucleotides. This will have the effect of enriching for signal and undercounting non-signal components of an iterative label signal, because of either of the correct iterative components for either of the target analytes comprises a signal at a temporal position, the merged iterative label will register a signal rather than a non-signal. Accordingly, merged iterative labels arising from target analytes situated beyond (that is, within) a diffraction limit for detection will be enriched for signal over non-signal components. Accordingly, by designing iterative labels for a probe population that have, for example no more than 50% signal components, one can confidently identify a merged iterative label if it comprises more than 50% signal components. Thus, by employing a signal-depleted iterative label threshold for each member of a probe population, one may readily identify merged iterative labels as having a signal contribution to the iterative label of above the threshold. In some cases, a probe population has no more than 70%, 65%, 60%, 55%, 50%, 45%, 40%, 35%, 30%, 25%, 20% or less than 20% signal components. In some cases a probe population has a fixed signal contribution to the iterative label that is uniform among members of the population, or thar varies within a fixed range, such that iterative labels having signal contributions above the fixed uniform level or greater than the fixed range, is identified as arising from not one but more than one target analyte signal. Alternately, iterative labels having a signal contribution above a threshold of, for example 95%, 90%, 85%, 80%, 75%, 70%, 65%, or 50% are identified as arising from not one but more than one target analyte signal.

In an alternate quality control approach, code words are selected for target analytes that are suspected of co-localizing such that their overlapping signals form distinct ‘merged’ words that are nonetheless unique and sufficient to identify both target analytes at a position, despite the target analytes being adjacent within a diffraction limit precluding their distinct detection in a single detection round.

In some cases, these approaches comprise assigning code words to individual transcripts having low signal/non-signal proportions, such that simultaneous reading of the code words may often place signal for a first word where non-signal is assigned for the second word. The merged code word will have a substantially higher proportion of signal to non-signal, but will nonetheless be unique for a sample or sufficient to distinguish themselves from other code words in the sample.

This approach is particularly useful when loci are jointly implicated in translocations or gene fusions, such as those that occur pursuant to some cancers. An incomplete list of examples of oncogenic fusion genes or translocations include BRC-ABL, TEL-AML, AML1-ETO, BCAM-AKT2, and TMPRSS2-ERG, and other fusion mentioned herein.

### High Resolution Datasets

Disclosed herein are high resolution images of data representative of analytes collected from a sample such as a two-dimensional sample section. The images may represent at least 100, at least 200, at least 500, at least 1,000, at least 2,000, at least 5,000, at least 10,000 or more than 10,000 analytes. The images of data representative of analytes preserve relative positional information of the analytes with respect to one another. In some cases the data representative of analytes superimposed upon an image of the sample from which the analyte data are collected. Images are high resolution in that at least some of the analytes in the high resolution images are positioned in close proximity to one another, for example less than 10 um, less than 9 um, less than 8 um, less than 7 um, less than 6 um, less than 5 um, less than 4 um, less than 3 um, less than 2 um, or less than 1 um from one another. In some cases analytes are positioned at distance from one another that is greater than (that is, below) the resolution limit of the wavelength of light used to capture the analyte data.

Data for the high resolution images, such as positional data, may in some cases be correlated to sequence data, such as sequence data corresponding to at least some of the analytes for which data is collected. Sequence data may be generated concurrently or may be assigned to analyte positions based upon annealing of probes of known sequence, among other approaches.

Data from high resolution images, such as positional data, may in some cases be assigned to individual cells of a sample. Analytes in a dataset are in some cases assigned to cells at a density of at least 200, 500, 1,000, 2,000, 5,000, 10,000 or more than 10,000 analytes per cell.

Data from the high resolution datasets are in some cases presented visually, such as on a computer monitor or on tangible medium such as paper. Visual presentation of a particular sample section is in some cases presented with or integrated with adjacent or related sample section data so as to reconstitute a three-dimensional depiction of analyte distributions in a reconstituted three-dimensional depiction of a sample. Such as depiction may be presented on a computer, on a tangible medium such as paper, or may be projected holographically, among other approaches for data presentation.

Data from high resolution datasets may be used to identify co-localized analytes within a sample. Analytes may be identified as co-localized if they occur at a resolution of, for example, less than 10 um, less than 9 um, less than 8 um, less than 7 um, less than 6 um, less than 5 um, less than 4 um, less than 3 um, less than 2 um, or less than 1 um from one another. In some cases analytes identified as co-localized if they are positioned at distance from one another that is greater than (that is, below) the resolution limit of the wavelength of light used to capture the analyte data. In some cases analytes identified as co-localized are indicated to a user. Indication may be internal to images of the high resolution datasets, such as by coloration or other visual emphasis of co-localization positions. Alternately or in combination, indication may be parallel to image generation, and may constitute for example FastA or other non-visual presentation of data, such as a list. Such a list may be compared to known analyte co-localization events, such as may occur when genomic translocations result in chimeric fusion transcripts that colocalize otherwise distinct transcripts or portions of otherwise distinct transcripts. Alternately, colocalization information for distinct analytes may suggest as a hypothesis that two analytes such as transcripts are fused in a single transcript in the sample. Such an hypothesis may be independently assayed using molecular biological techniques, such as a reverse-transcription PCR assay using inward facing primers from each of the putative fusion transcripts, where amplification may independently confirm co-localization and further suggest that a fusion even has occurred.

Data for high resolution datasets, and the images generated therefrom, may be generated through a number of approaches disclosed herein. Some approaches comprise at least two instances of iterative hybridizations of probe sets targeting analytes in the sample to form code words indicative of analyte localization, such that the signals generated from the probe sets may be superimposed. Adjacent or entirely overlapping signals from temporally distinct detection events may be indicative of analytes that are adjacent in the sample, at a separation of for example, less than 10 um, less than 9 um, less than 8 um, less than 7 um, less than 6 um, less than 5 um, less than 4 um, less than 3 um, less than 2 um, or less than 1 um from one another. In some cases analytes are identified as co-localized if they are positioned at distance from one another that is greater than (that is, below) the resolution limit of the wavelength of light used to capture the analyte data. Alternately or in combination, data for high resolution datasets, and the images generated therefrom, may be generated through sequencing of nucleic acids in sample sections or tagged to probes binding analytes in the samples. Sequencing may be effected through a broad range of approaches, such as contacting the sample to position-tagged beads of less than 10 um in diameter, or other approaches in the art.

Data for the high resolution images are captured by using substantially fewer wavelengths than are analytes to be analyzed. For example, a plurality of analytes (such as at least 100, at least 200, at least 500, at least 1,000, at least 2,000, at least 5,000, at least 10,000 or more than 10,000 analytes) are assayed by capturing emission spectra of no more than 10, 9, 8, 7, 6, 5, 4, 3, or 2 wavelengths, or of a single wavelength. In many exemplary embodiments the plurality of analytes are assayed using no more than 4, no more than 3, or no more than 2 wavelengths. Similarly, data for the high resolution images is in some cases captured in a device having no more than 4 channels of light detection, no more than 3 channels of light detection, or no more than 2 channels of light detection. Analytes are identified in may approaches not by a single emission signal, but by a series of emission signals that temporally form a ‘word’ indicative or, specifying, or unique to the analyte.

### METHODS AND EXAMPLES

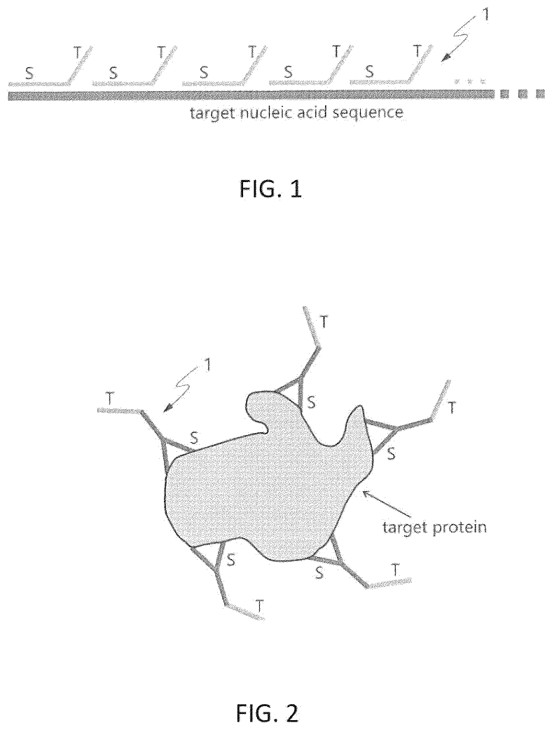

In an application variant, the analyte or target is nucleic acid, e.g., DNA or RNA, and the probe set comprises oligonucleotides that are partially or completely complementary to the whole sequence or a subsequence of the nucleic acid sequence to be detected (FIG. 1). The nucleic acid sequence specific oligonucleotide probe sets comprising analyte-specific probes (1) including a binding element(S) that specifically hybridizes to the target nucleic acid sequence to be detected, and an identifier element (T) comprising a nucleotide sequence which is unique to said set of analyte-specific probes (unique identifier sequence).

In a further application variant, the analyte or target is a protein, and the probe set comprises one or more proteins, e.g. antibodies (FIG. 2). The protein specific probe set comprising analyte-specific probes (1) including a binding element (T) such as the (hyper-) variable region of an antibody, that specifically interacts with the target protein to be detected, and the identifier element (T).

In a further application variant, at least one analyte is a nucleic acid and at least a second analyte is a protein and at least the first probe set binds to the nucleic acid sequence and at least the second probe set binds specifically to the protein analyte. Other combinations are possible as well.

An embodiments of the general method of the present disclosure may be:
Step 1: Applying the at least 20 analyte-or target-specific probe sets. The target nucleic acid sequence is incubated with a probe set consisting of oligonucleotides with sequences complementary to the target nucleic acid. In this example, a probe set of 5 different probes is shown, each comprising a sequence element complementary to an individual subsequence of the target nucleic acid sequence (S1 to S5). In this example, the regions do not overlap. Each of the oligonucleotides targeting the same nucleic acid sequence comprises the identifier element or unique identifier sequence (T), respectively.
Step 2: Hybridization of the probe set. The probe set is hybridized to the target nucleic acid sequence under conditions allowing a specific hybridization. After the incubation, the probes are hybridized to their corresponding target sequences and provide the identifier element (T) for the next steps.
Step 3: Eliminating non-bound probes. After hybridization, the unbound oligonucleotides are eliminated, e.g., by washing steps.
Step 4: Applying the decoding oligonucleotides. The decoding oligonucleotides consisting of at least two sequence elements (t) and (c) are applied. While sequence element (t) is complementary to the unique identifier sequence (T), the sequence element (c) provides a region for the subsequent hybridization of signal oligonucleotides (translator element).
Step 5: Hybridization of decoding oligonucleotides. The decoding oligonucleotides are hybridized with the unique identifier sequences of the probes (T) via their complementary first sequence elements (t). After incubation, the decoding oligonucleotides provide the translator sequence element (c) for a subsequent hybridization step.
Step 6: Eliminating the excess of decoding oligonucleotides. After hybridization, the unbound decoding oligonucleotides are eliminated, e.g., by washing steps.
Step 7: Applying the signal oligonucleotide. The signal oligonucleotides are applied. The signal oligonucleotides comprise at least one second connector element (C) that is essentially complementary to the translator sequence element (c) and at least one signal element that provides a detectable signal (F).
Step 8: Hybridization of the signal oligonucleotides. The signal oligonucleotides are hybridized via the complementary sequence connector element (C) to the translator element (c) of decoding oligonucleotide. After incubation, the signal oligonucleotides are hybridized to their corresponding decoding oligonucleotides and provide a signal (F) that can be detected.
Step 9: Eliminating the excess of signal oligonucleotides. After hybridization, the unbound signal oligonucleotides are eliminated, e.g., by washing steps.
Step 10: Signal detection. The signals provided by the signal oligonucleotides are detected.
The following steps (steps 11 and 12) are unnecessary for the last detection round.
Step 11: Selective denaturation. The hybridization between the unique identifier sequence (T) and the first sequence element (t) of the decoding oligonucleotides is dissolved. The destabilization can be achieved via different mechanisms well known to the trained person like for example: increased temperature, denaturing agents, etc. The target-or analyte-specific probes are not affected by this step.
Step 12: Eliminating the denatured decoding oligonucleotides. The denatured decoding oligonucleotides and signal oligonucleotides are eliminated (e.g., by washing steps) leaving the specific probe sets with free unique identifier sequences, reusable in a next round of hybridization and detection (steps 4 to 10). This detection cycle (steps 4 to 12) is repeated at least four times until the planed encoding scheme is completed.

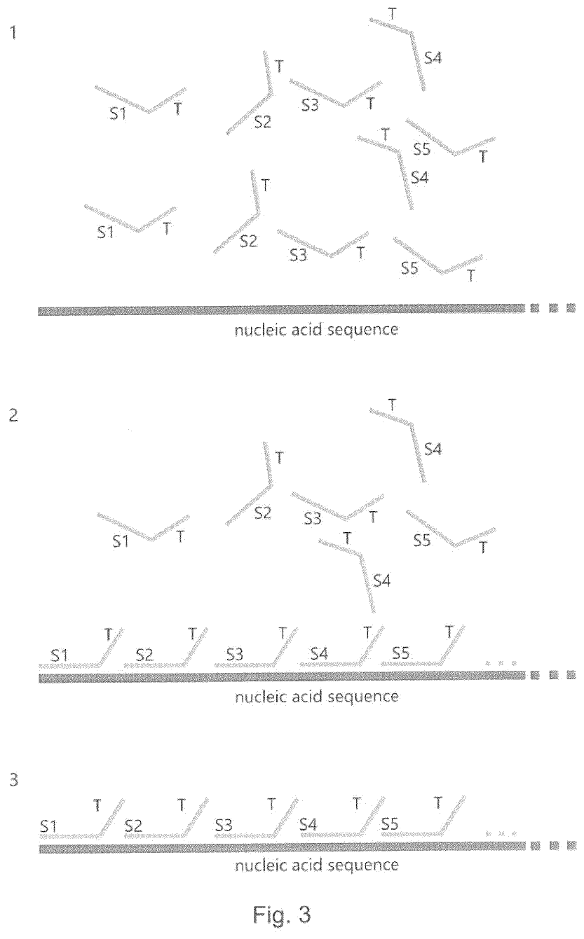

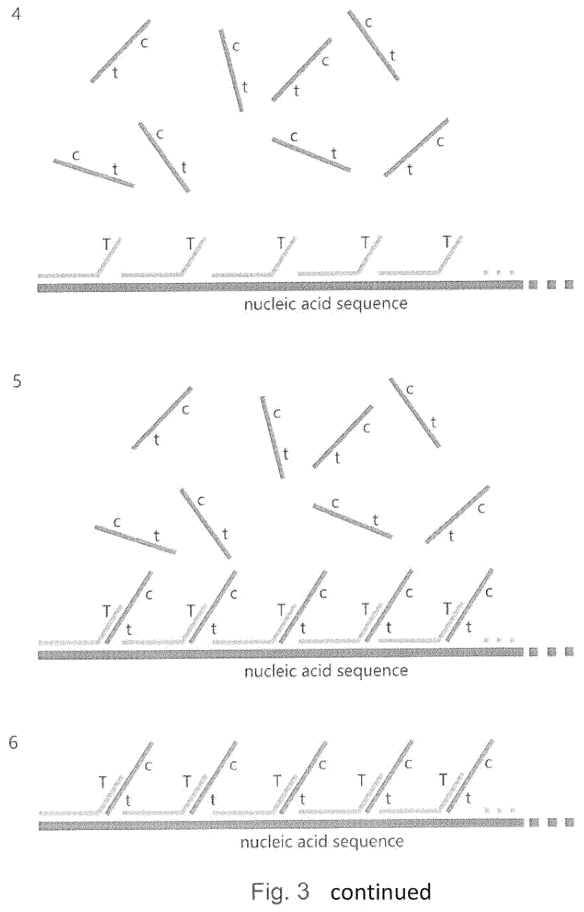

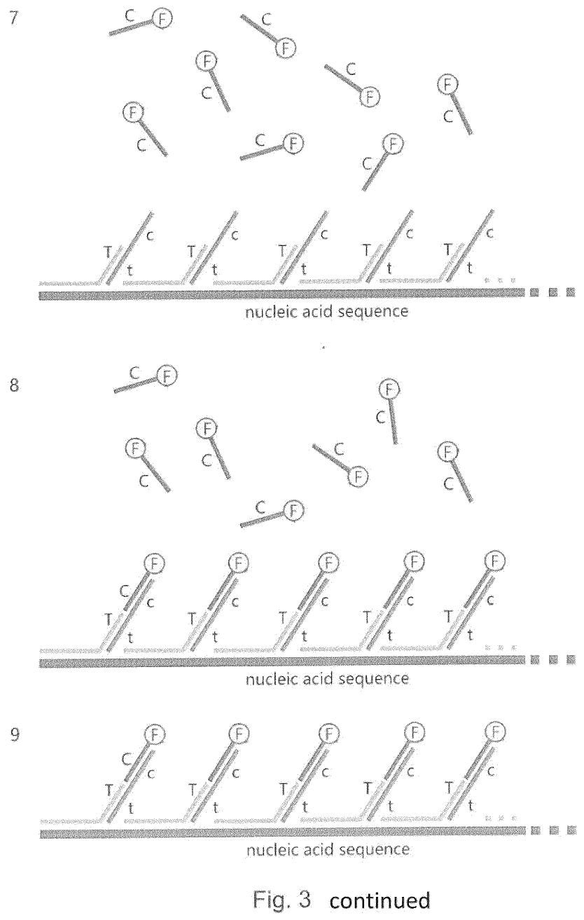

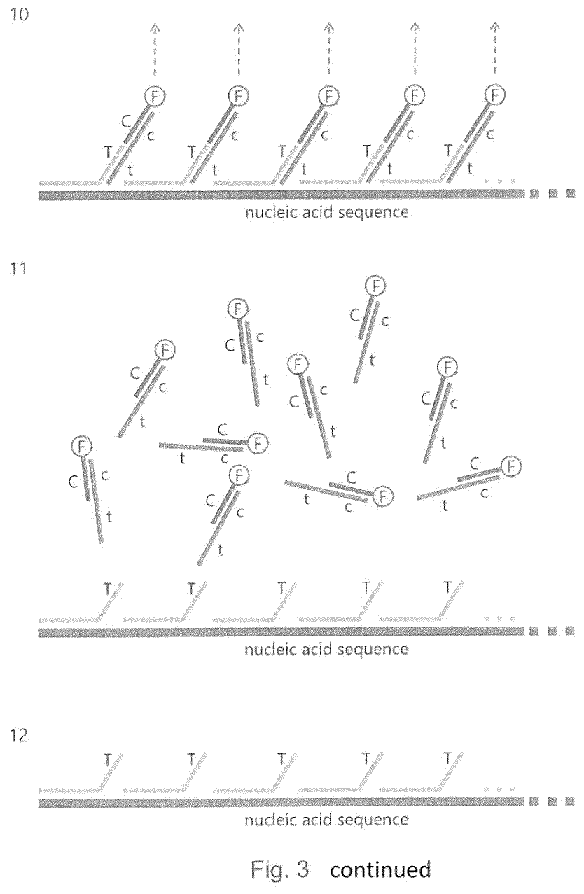

Another embodiment of the general method of the present disclosure using multi-decoders may be (FIG. 16):
Step 1: Target nucleic acids: In this example three different target nucleic acids (A), (B) and (C) have to be detected and differentiated by using only two different types of signal oligonucleotides. Before starting the experiment, a certain encoding scheme is set. In this example, the three different nucleic acid sequences are encoded by three rounds of detection with three different signal types (1), (2) and (½) and a resulting hamming distance of 3 to allow for error detection. The planed code words are:
sequence A: (1)-(1)-(2)
sequence B: (2)-(2)-(½)
sequence C: (½)-(½)-(1)
Step 2: Hybridization of the probe sets: For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (A) is labeled with (T1), sequence (B) with (T2) and sequence (C) with (T3). The illustration in FIG. 16 summarizes Steps 1 to 3 of FIG. 3.
Step 3: Hybridization of the decoding oligonucleotides and multi-decoders: For each unique identifier present, a certain decoding oligonucleotide or multi-decoder is applied specifically hybridizing to the corresponding unique identifier sequence by its first sequence element (here (t1) to (T1), (t2) to (T2) and (t3) to (T3)). Each of the decoding oligonucleotides or multi-decoders provides a translator or two translator elements that define the signals that will be generated after hybridization of signal oligonucleotides. Here nucleic acid sequence (A) is labeled with (c1), (B) is labeled with (c2) and (C) is labeled with both translator elements (c1) and (c2) resulting in the signal (½). The illustration in FIG. 16 summarizes steps 4 to 6 of FIG. 3.
Step 4: Hybridization of signal oligonucleotides: For each type of translator element, a signal oligonucleotide with a certain signal, differentiable from signals of other signal oligonucleotides, is applied. This signal oligonucleotide can specifically hybridize to the corresponding translator element. The illustration in FIG. 16 summarizes steps 7 to 9 of FIG. 3
Step 5: Signal detection for the encoding scheme: The different signals are detected. Note that in this example the nucleic acids (A), (B) and (C) can already be distinguished after the first round of detection. This is in contrast to the step 5 of FIG. 5 explained by the additional signal type (½) that can be realized due to multi-decoders. Although nucleic acid sequences can already be distinguished, the additional rounds contribute to the planned hamming distance of 3. The illustration in FIG. 16 corresponds to step 10 of FIG. 3.
Step 6: Selective denaturation: The decoding (and signal) oligonucleotides and/or multi-decoders of all nucleic acid sequences to be detected are selectively denatured and eliminated as described in steps 11 and 12 of FIG. 3. Afterwards the unique identifier sequences of the different probe sets can be used for the next round of hybridization and detection.
Step 7: Second round of detection: A next round of hybridization and detection is done as described in steps 3 to 5. Note that in this new round the mix of different decoding oligonucleotides and multi-decoders is changed. For example, decoding oligonucleotide of nucleic acid sequence (A) used in the first round comprised of sequence elements (t1) and (c1) while the new multi decoder of round 2 comprises of the sequence elements (t1), (c1) and (c2). Note that now a hamming distance of 2 is already given after 2 rounds, which is the final result of the example in FIG. 3 after 3 rounds.
Step 8: Third round of detection: Again, a new combination of decoding oligonucleotides and/or multi-decoders is used leading to new signal combinations. After signal detection, the resulting code words for the three different nucleic acid sequences are not only unique and therefore distinguishable but comprise a hamming distance of 3 to other code words. Due to the hamming distance, an error in the detection of the signals (signal exchange) would not result in a valid code word and therefore could be detected and because of hamming distance 3 also corrected, in contrast to the encoding scheme of FIG. 3. This way three different nucleic acids can be distinguished in three detection rounds with two different signals, allowing an error detection and correction.

Note that in every round of detection, the type of signal provided by a certain unique identifier is controlled by the use of a certain decoding oligonucleotide. As a result, the sequence of decoding oligonucleotides applied in the detection cycles transcribes the binding specificity of the probe set into a unique signal sequence.

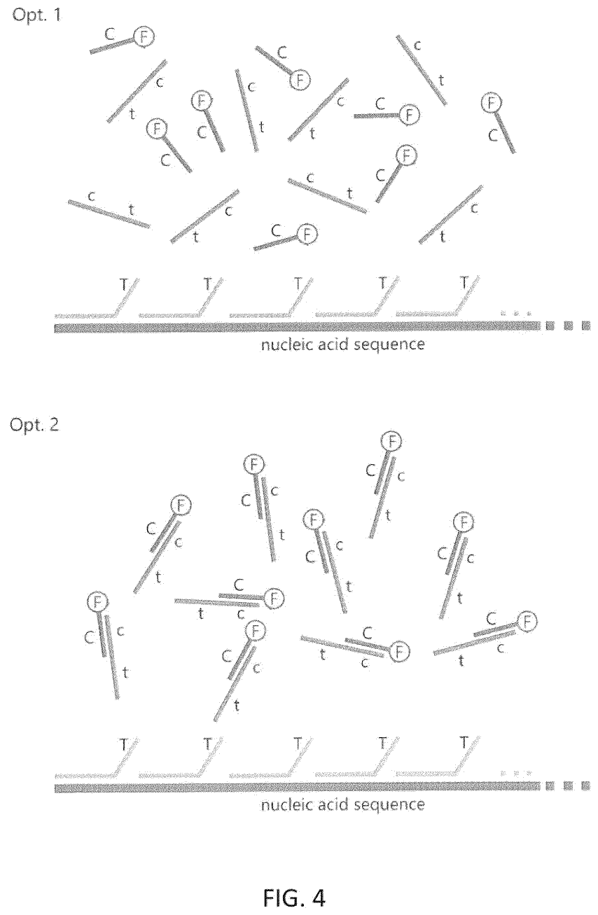

The steps of decoding oligonucleotide hybridization (steps 4 to 6) and signal oligonucleotide hybridization (steps 7 to 9) can also be combined in two alternative ways as shown in FIG. 4.

Opt. 1: Simultaneous hybridization. Instead of the steps 4 to 9 of FIG. 3, specific hybridization of decoding oligonucleotides and signal oligonucleotides can also be done simultaneously leading to the same result as shown in step 9 of FIG. 3, after eliminating the excess decoding- and signal oligonucleotides.

Opt. 2: Preincubation. Additionally, to option 1 of FIG. 3, decoding- and signal oligonucleotides can be preincubated in a separate reaction before being applied to the target nucleic acid with the already bound specific probe set.

### 1. Example for Signal Encoding of Three Different Nucleic Acid Sequences by Two Different Signal Types and Three Detection Rounds

FIG. 3 shows the general concept of generation and detection of specific signals mediated by decoding oligonucleotides. It does not show the general concept of encoding that can be achieved by this procedure. To illustrate the use of the process shown in FIG. 3 for the generation of an encoding scheme, FIG. 5 shows a general example for a multiple round encoding experiment with three different nucleic acid sequences. In this example, the encoding scheme includes error detection.

Step 1: Target nucleic acids. In this example three different target nucleic acids (A), (B) and (C) have to be detected and differentiated by using only two different types of signals. Before starting the experiment, a certain encoding scheme is set. In this example, the three different nucleic acid sequences are encoded by three rounds of detection with two different signals (1) and (2) and a resulting hamming distance of 2 to allow for error detection. The planed code words are:
sequence A: (1)-(2)-(2);
sequence B: (1)-(1)-(1);
sequence C: (2)-(1)-(2).

Step 2: Hybridization of the probe sets. For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (T) is labeled with (T1), sequence (B) with (T2) and sequence (C) with (T3). The illustration summarizes steps 1 to 3 of FIG. 3.

Step 3: Hybridization of the decoding oligonucleotides. For each unique identifier present, a certain decoding oligonucleotide is applied specifically hybridizing to the corresponding unique identifier sequence by its first sequence element (here (t1) to (T1), (t2) to (T2) and (t3) to (T3)). Each of the decoding oligonucleotides provides a translator element that defines the signal that will be generated after hybridization of signal oligonucleotides. Here nucleic acid sequences (A) and (B) are labeled with the translator element (c1) and sequence (C) is labeled with (c2). The illustration summarizes steps 4 to 6 of FIG. 3.

Step 4: Hybridization of signal oligonucleotides. For each type of translator element, a signal oligonucleotide with a certain signal (2), differentiable from signals of other signal oligonucleotides, is applied. This signal oligonucleotide can specifically hybridize to the corresponding translator element. The illustration summarizes steps 7 to 9 of FIG. 3.

Step 5: Signal detection for the encoding scheme. The different signals are detected. Note that in this example the nucleic acid sequence (C) can be distinguished from the other sequences by the unique signal (2) it provides, while sequences (A) and (B) provide the same kind of signal (1) and cannot be distinguished after the first cycle of detection. This is due to the fact, that the number of different nucleic acid sequences to be detected exceeds the number of different signals available. The illustration corresponds to step 10 of FIG. 3.

Step 6: Selective denaturation. The decoding (and signal) oligonucleotides of all nucleic acid sequences to be detected are selectively denatured and eliminated as described in steps 11 and 12 of FIG. 3.

Afterwards the unique identifier sequences of the different probe sets can be used for the next round of hybridization and detection.

Step 7: Second round of detection. A next round of hybridization and detection is done as described in steps 3 to 5. Note that in this new round the mix of different decoding oligonucleotides is changed. For example, decoding oligonucleotide of nucleic acid sequence (A) used in the first round comprised of sequence elements (t1) and (c1) while the new decoding oligonucleotide comprises of the sequence elements (t1) and (c2). Note that now all three sequences can clearly be distinguished due to the unique combination of first and second round signals.

Step 8: Third round of detection. Again, a new combination of decoding oligonucleotides is used leading to new signal combinations. After signal detection, the resulting code words for the three different nucleic acid sequences are not only unique and therefore distinguishable but comprise a hamming distance of 2 to other code words. Due to the hamming distance, an error in the detection of the signals (signal exchange) would not result in a valid code word and therefore could be detected. By this way three different nucleic acids can be distinguished in three detection rounds with two different signals, allowing error detection.

### 2. Advantages Over Prior Art Technologies

### Coding Strategy

Compared to state-of-the-art methods, one particular advantage of the method according to the disclosure is the use of decoding oligonucleotides breaking the dependencies between the target specific probes and the signal oligonucleotides.

Without decoupling target specific probes and signal generation, two different signals can only be generated for a certain target if using two different molecular tags. Each of these molecular tags can only be used once. Multiple readouts of the same molecular tag do not increase the information about the target. In order to create an encoding scheme, a change of the target specific probe set after each round is required (SeqFISH) or multiple molecular tags must be present on the same probe set (like merFISH, intronSeqFISH).

Following the method according to the disclosure, different signals are achieved by using different decoding oligonucleotides reusing the same unique identifier (molecular tag) and a small number of different, mostly cost-intensive signal oligonucleotides. This leads to several advantages in contrast to the other methods.
(1) The encoding scheme is not defined by the target specific probe set as it is the case for all other methods of prior art. Here the encoding scheme is transcribed by the decoding oligonucleotides. This leads to a much higher flexibility concerning the number of rounds and the freedom in signal choice for the codewords. Looking on the methods of prior art (e.g., merFISH or intronSeqFISH), the encoding scheme (number, type and sequence of detectable signals) for all target sequences is predefined by the presence of the different tag sequences on the specific probe sets (4 of 16 different tags per probe set in the case of merFISH and 5 of 60 different tags in the case of intron FISH). In order to produce a sufficient number of different tags per probe set, the methods use rather complex oligonucleotide designs with several tags present on one target specific oligonucleotide. In order to change the encoding scheme for a certain target nucleic acid, the specific probe set has to be replaced. The method according to the disclosure describes the use of a single unique tag sequence (unique identifier) per analyte, because it can be reused in every detection round to produce a new information. The encoding scheme is defined by the order of decoding oligonucleotides that are used in the detection rounds. Therefore, the encoding scheme is not predefined by the specific probes (or the unique tag sequence) but can be adjusted to different needs, even during the experiment. This is achieved by simply changing the decoding oligonucleotides used in the detection rounds or adding additional detection rounds.
(2) The number of different signal oligonucleotides must match the number of different tag sequences with methods of prior art (16 in the case of merFISH and 60 in the case of intronSeqFISH). Using the method according to the disclosure, the number of different signal oligonucleotides matches the number of different signals used. Due to this, the number of signal oligonucleotides stays constant for the method described here and never exceeds the number of different signals but increases with the complexity of the encoding scheme in the methods of prior art (more detection rounds more different signal oligonucleotides needed). As a result, the method described here leads to a much lower complexity (unintended interactions of signal oligonucleotides with environment or with each other) and dramatically reduces the cost of the assay since the major cost factor are the signal oligonucleotides.
(3) In the methods of prior art, the number of different signals generated by a target specific probe set is restricted by the number of different tag sequences the probe set can provide. Since each additional tag sequence increases the total size of the target specific probe, there is a limitation to the number of different tags a single probe can provide. This limitation is given by the size dependent increase of several problems (unintended inter- and intramolecular interactions, costs, diffusion rate, stability, errors during synthesis etc.). Additionally, there is a limitation of the total number of target specific probes that can be applied to a certain analyte. In case of nucleic acids, this limitation is given by the length of the target sequence and the proportion of suitable binding sites. These factors lead to severe limitations in the number of different signals a probe set can provide (4 signals in the case of merFISH and 5 signals in the case of intronSeqFISH). This limitation substantially affects the number of different code words that can be produced with a certain number of detection rounds. In the approach of the disclosure only one tag is needed and can be freely reused in every detection round. This leads to a low oligonucleotide complexity/length and at the same time to the maximum encoding efficiency possible (number of colors number of rounds). The vast differences of coding capacity of our method compared to the other methods is shown in FIGS. 1 and 5. Due to this in approach of the disclosure a much lower number of detection rounds is needed to produce the same amount of information. A lower number of detection rounds is connected to lower cost, lower experimental time, lower complexity, higher stability and success rate, lower amount of data to be collected and analyzed and a higher accuracy of the results.

### 3. Coding Capacity

All three methods compared in the Table 1 below use specific probe sets that are not denatured between different rounds of detection. For intronSeqFISH there are four detection rounds needed to produce the pseudo colors of one coding round, therefore data is only given for rounds 4, 8, 12, 16 and 20. The merFISH-method uses a constant number of 4 signals, therefore the data starts with the smallest number of rounds possible. After 8 detection rounds our method exceeds the maximum coding capacity reached with 20 rounds of merFISH (depicted with one asterisk) and after 12 rounds of detection the maximum coding capacity of intron FISH is exceeded (depicted with two asterisks).

For the method according to the disclosure usage of 3 different signals is assumed (as is with intronSeqFISH).

TABLE 1
Comparison of coding capacity

| | Coding capacity | | |
|---|---|---|---|
| | Method of | | |
| Number of | the present | intron | |
| Rounds: | disclosure | FISH | merFISH |
| 1 | 3 | — | — |
| 2 | 9 | — | — |
| 3 | 27 | — | — |
| 4 | 81 | 12 | 1 |
| 5 | 243 | — | 5 |
| 6 | 729 | — | 15 |
| 7 | 2187 | — | 35 |
| 8* | 6561 | 144 | 70 |
| 9 | 19683 | — | 126 |
| 10 | 59049 | — | 210 |
| 11 | 177147 | — | 330 |
| 12** | 531441 | 1728 | 495 |
| 13 | 1594323 | — | 715 |
| 14 | 4782969 | — | 1001 |
| 15 | 14348907 | — | 1365 |
| 16 | 43046721 | 20736 | 1820 |
| 17 | 129140163 | — | 2380 |
| 18 | 387420489 | — | 3060 |
| 19 | 1162261467 | — | 3876 |
| 20 | 3486784401 | 248832 | 4845 |

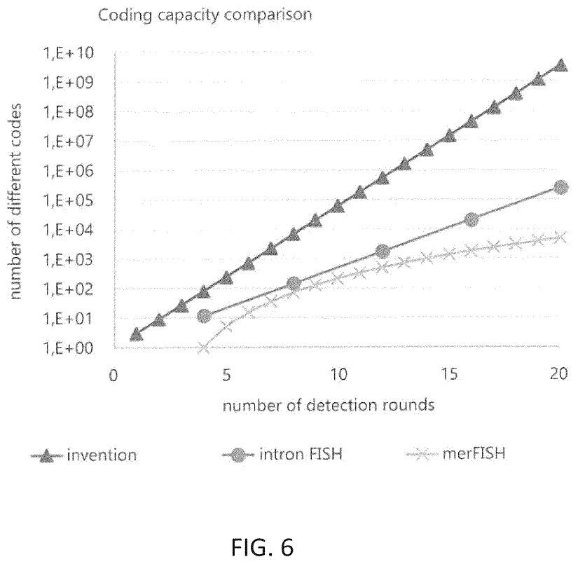

As shown in FIG. 6 the number of codewords for merFISH does not exponentially increase with the number of detection cycles but gets less effective with each added round. In contrast, the number of codewords for intronSeqFISH in the method according to the disclosure increases exponentially. The slope of the curve for the proposed method is much higher than that of intron FISH, leading to more than 10,000 times more code words usable after 20 rounds of detection.

Note that this maximum efficiency of coding capacity is also reached in case of seqFISH, where specific probes are denatured after every detection round and a new probe set is specifically hybridized to the target sequence for each detection round. However, this method has major downsides to technologies using only one specific hybridization for their encoding scheme (all other methods):
(1) For the efficient denaturation of the specific probes, rather crude conditions must be used (high temperatures, high concentrations of denaturing agent, long incubation times) leading to much higher probability for the loss or the damage of the analyte.
(2) For each round of detection an own probe set has to be used for every target nucleic acid sequence. Therefore, the number of specific probes needed for the experiment scales with the number of different signals needed for the encoding scheme. This dramatically increases the complexity and the cost of the assay.
(3) Because the hybridization efficiency of every target nucleic acid molecule is subject to some probabilistic effects, the fluctuations of signal intensity between the different detection rounds is much higher than in methods using only one specific hybridization event, reducing the proportion of complete codes.
(4) The time needed for the specific hybridization is much longer than for the hybridization of signal oligonucleotides or decoding oligonucleotides (as can be seen in the method parts of the intronSeqFISH, merFISH and seqFISH publications), which dramatically increases the time needed to complete an experiment.

Due to these reasons all other methods use a single specific hybridization event and accept the major downside of lower code complexity and therefore the need of more detection rounds and a higher oligonucleotide design complexity.

The method according to the disclosure combines the advantages of seqFISH (mainly complete freedom concerning the encoding scheme) with all advantages of methods using only one specific hybridization event while eliminating the major problems of such methods.

Note that the high numbers of code words produced after 20 rounds can also be used to introduce higher hamming distances (differences) between different codewords, allowing error detection of 1, 2 or even more errors and even error corrections. Therefore, even very high coding capacities are still practically relevant.

As mentioned above, the usage of multi-decoders further increases the coding capacity of the encoding scheme. Instead of being limited to the having exactly the same number of different signal types as different signal oligonucleotides and corresponding translator elements, the use of multi-decoders increases the signal types that can be used to: (N×(N+1))/2 (with N being the number of different signal oligonucleotides used). For the code used in table 1 with 3 different signal oligonucleotides this would mean the following 7 different signal types could be used: 10 (S1), (S2), (S3), (S1/S2), (S1/S3), (S2/S3), (S1/S2/S3). The effect to the coding efficiency can be seen in Table 1b and FIG. 17.

| | Coding capacity | | | |
|---|---|---|---|---|
| | | Method of | | |
| | | the present | | |
| | Method of | disclosure | | |
| Number of | the present | with multi- | intron | |
| Rounds: | disclosure | decoders | FISH | merFISH |
| 1 | 3 | 7 | — | — |
| 2 | 9 | 49 | — | — |
| 3 | 27 | 343 | — | — |
| 4 | 81 | 2401 | 12 | 1 |
| 5* | 243 | 16807 | — | 5 |
| 6 | 729 | 117649 | — | 15 |
| 7** | 2187 | 823543 | — | 35 |
| 8 | 6561 | 5764801 | 144 | 70 |
| 9 | 19683 | 40353607 | — | 126 |
| 10 | 59049 | 282475249 | — | 210 |
| 11 | 177147 | 1977326743 | — | 330 |
| 12*** | 531441 | 1,3841 × 10\hat{ }10 | 1728 | 495 |
| 13 | 1594323 | 9,6889 × 10\hat{ }10 | — | 715 |
| 14 | 4782969 | 6,7822 × 10\hat{ }11 | — | 1001 |
| 15 | 14348907 | 4,7476 × 10\hat{ }12 | — | 1365 |
| 16 | 43046721 | 3,3233 × 10\hat{ }13 | 20736 | 1820 |
| 17 | 129140163 | 2,3263 × 10\hat{ }14 | — | 2380 |
| 18 | 387420489 | 1,6284 × 10\hat{ }15 | — | 3060 |
| 19 | 1162261467 | 1,1399 × 10\hat{ }16 | — | 3876 |
| 20 | 3486784401 | 7,9792 × 10\hat{ }16 | 248832 | 4845 |

Table 1b shows the coding capacity of the four methods. All four methods compared in the table use specific probe sets that are not denatured between different rounds of detection. For intronSeqFISH there are four detection rounds needed to produce the pseudo colors of one coding round, therefore data is only given for rounds 4, 8,12,16 and 20. The merFISH-method uses a constant number of 4 signals, therefore the data starts with the smallest number of rounds possible. After 4 detection rounds the method with multi-decoders as described here exceeds the maximum coding capacity reached with 20 rounds of merFISH (depicted with one asterisk), after 7 rounds of detection the maximum coding capacity of intron FISH is exceeded (depicted with two asterisks) and after 12 rounds of detection the maximum coding capacity of the method of the present disclosure is exceeded (depicted with three asterisks). The usage of 3 different signal oligonucleotides is assumed (as is with intronSeqFISH).

### 4. Selective Denaturation, Oligonucleotide Assembly and Reuse of Unique Identifiers are Surprisingly Efficient

A key factor of the method according to the disclosure is the consecutive process of decoding oligonucleotide binding, signal oligonucleotide binding, signal detection and selective denaturation. In order to generate an encoding scheme, this process has to be repeated several times (depending on the length of the code word). Because the same unique identifier is reused in every detection cycle, all events from the first to the last detection cycle are depending on each other. Additionally, the selective denaturation depends on two different events: While the decoding oligonucleotide has to be dissolved from the unique identifier with highest efficiency, specific probes have to stay hybridized with highest efficiency.

Due to this the efficiency E of the whole encoding process can be described by the following equation:

$$E = {B}_{s p} \times {( {B}_{d e} \times {B}_{s i} \times {E}_{d e} \times {S}_{s p} )}^{n}$$

E=total efficiency
Bₛₚ=binding of specific probes
B_{de}=binding of decoding oligonucleotides
Bₛᵢ=binding of signal oligonucleotides
E_{de}=elimination of decoding oligonucleotides
Sₛₚ=stability of specific probes during elimination process
n=number of detection cycles

Based on this equation the efficiency of each single step can be estimated for a given total efficiency of the method. The calculation is hereby based on the assumption, that each process has the same efficiency. The total efficiency describes the portion of successfully decodable signals of the total signals present.

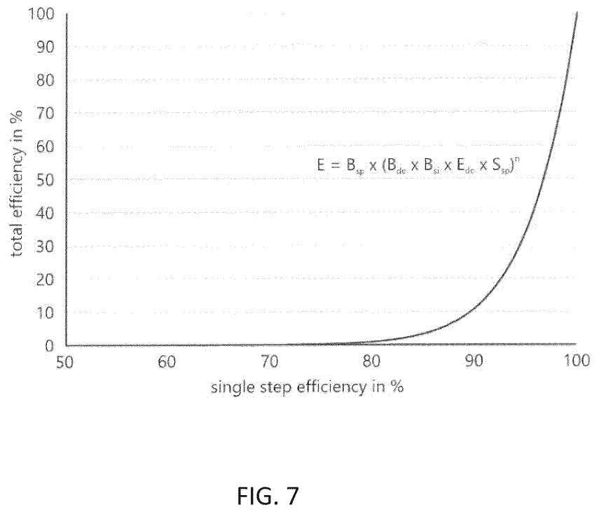

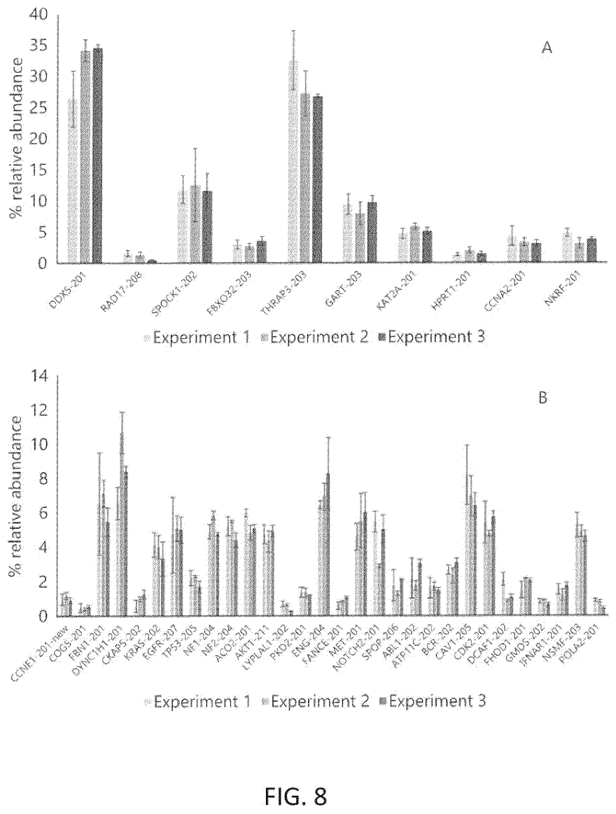

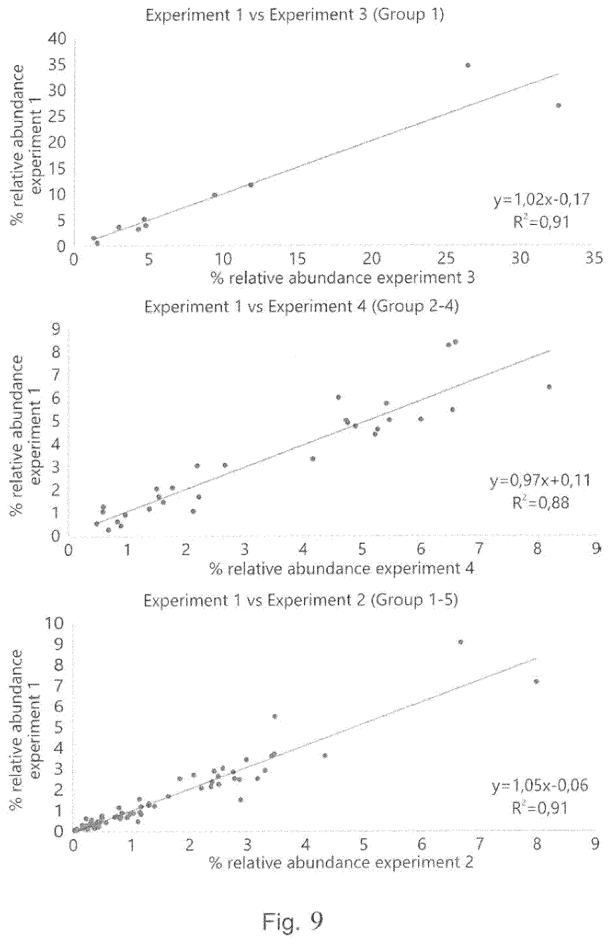

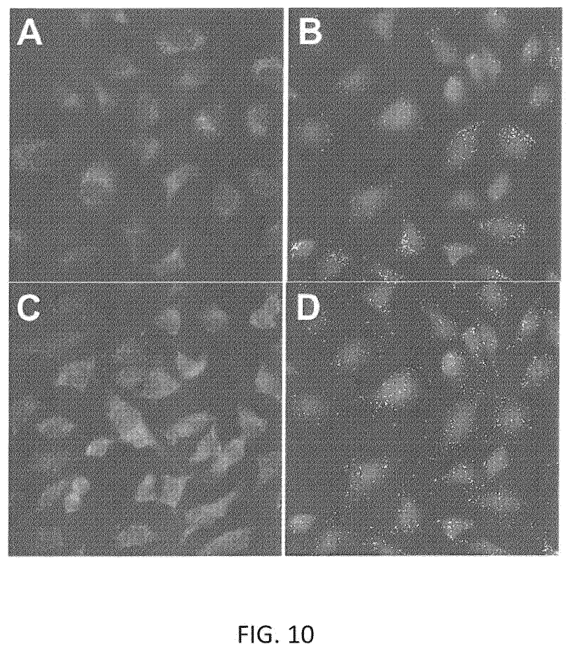

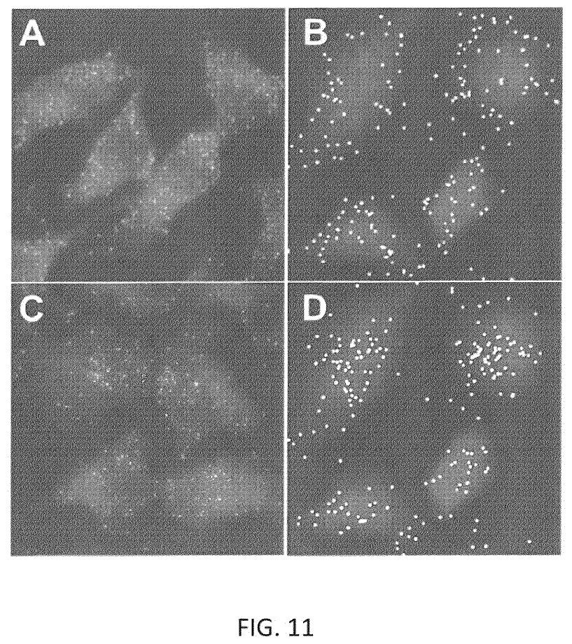

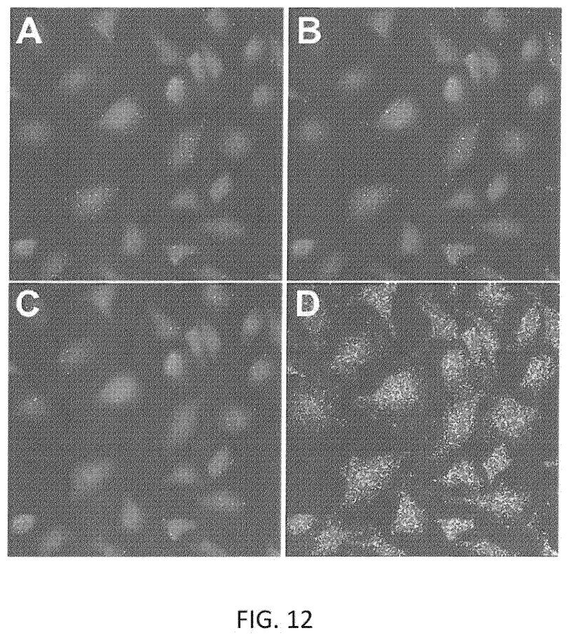

The total efficiency of the method is dependent on the efficiency of each single step of the different factors described by the equation. Under the assumption of an equally distributed efficiency the total efficiency can be plotted against the single step efficiency as shown in FIG. 7. As can be seen, a practically relevant total efficiency for an encoding scheme with 5 detection cycles can only be achieved with single step efficiencies clearly above 90%. For example, to achieve a total efficiency of 50% an average efficiency within each single step of 97.8% is needed. These calculations are even based on the assumption of a 100% signal detection and analysis efficiency. Due to broad DNA melting curves of oligonucleotides of a variety of sequences, the inventors assumed prior to experiments that the selective denaturation would work less efficient for denaturation of decoding oligonucleotides and that sequence specific binding probes are not stable enough. In contrast to this assumption, we found a surprising effectiveness of all steps and a high stability of sequence specific probes during selective denaturation.

Experimentally, the inventors achieved a total decoding efficiency of about 30% to 65% based on 5 detection cycles. A calculation of the efficiency of each single step (Bsp, Bde, Bsi, Ede, Ssp) by the formula given above revealed an average efficiency of about 94.4% to 98%. These high efficiencies are very surprising and cannot easily be anticipated by a well-trained person in this field.

### 5. Further Experimental Data

1. Example for signal encoding of two subgroups of nucleic acid sequences with spatial overlap The table below shows examples of implementations using two subgroups (analytic sets) in consecutive runs:

| | | Transcript | | | | |
|---|---|---|---|---|---|---|
| | | Plexity | Run 1 | | Run 2 | |
| | Total | (Set 1/ | Fluidics | Plexity | Fluidics | Plexity |
| | rounds | Set 2) | rounds | 1 | rounds | 2 |
| 1.0 | 9 | 100/2 | 8 | 100 (129) | 1 | 2 |
| 2.0 | 16 | 300/25 | 10 | 300 (419) | 6 | 25 (29) |
| 2.1 | 16 | 500/50 | 10 | 500 (685) | 6 | 50 (60) |
| Improved | | | | | | |
| transcript | | | | | | |
| detection | | | | | | |

For “run 1.0” n=8 rounds of hybridization and imaging was used to detect 100 genes. For “run 2.0” the number of rounds is increased to n=10 and can thereby target 300 genes. For “run 2.1” an enhanced transcript detection probe is used which allows to target 500 transcripts with the same number of rounds (n=10).

Consecutive runs are run on the same tissue sections to detect transcripts beyond the diffraction limit.

The main steps of the method are:
i. Hybridization of target probes of the two analytic sets happen simultaneously before the first run.
ii. Tails of the target probes are unique for both runs. e.g., 100, 300 and 500 tails (for 100, 300, or 500 transcripts, respectively) are used for the first run with the first analytic set and 2, 25, 50 tails (for 2, 25, 50 transcripts, respectively) for the second run with the second analytic set. This requires also different decoder sets for both runs.
iii. Both runs generate independent datasets that can be combined in-silico afterwards.

Thereby, 1) enhancing the multiplexing capability without increasing optical crowding and 2) detecting spatial overlapping transcripts which would be otherwise not possible due to the diffraction limit of the microscope. This is e.g., allows for detection of fusion genes (cancer) or for detection of co-localization of different transcript types (e.g. transcriptional hubs in the nucleus). In addition, transcripts with higher expression levels can also be analyzed because the “signal spread” of the high number of abundant signals vs. the detection of other (especially lowly expressed) genes is improved.

In principle, both runs can be also interwoven, e.g.:
Detection round 1=1st round of detection of first analytical set
Detection round 2=1st round of detection of second analytical set
Detection round 3=2nd round of detection of first analytical set
Detection round 4=2nd round of detection of second analytical set
Etc.

### 6. Example—Quality Control and Defeating the Diffraction Limit

The BCAM-AKT2 fusion is implicated in cancer progression. In a first run, a tissue is assayed for BCAM and AKT2 transcript localization. Code words are assigned to each target analyte, with 1 representing signal and 0 representing non-signal. No target analyte is assigned a code word having greater than 60% signal. BCAM is assigned a code word of 1001001, while AKT2 is assigned a code word of 0100100.

A sample tissue is assayed for BCAM and AKT2 localization. The sample harbours a BCAM-AKT2 fusion, resulting in BCAM probe sets and AKT2 probe sets binding to targets that are adjacent within a diffraction limit precluding their distinct detection in a single detection round.

The code word 1101101 is observed at multiple loci. The code word comprises 5/7 signal letters and 2/7 non-signal letters. This proportion is above a 60% threshold, and the word is identified as indicative of probe sets binding to targets that are adjacent within a diffraction limit precluding their distinct detection in a single detection round. However, the identity of the target at that location is not determined.

In a second run, a tissue is assayed for BCAM and AKT2 transcript localization. BCAM probe sets and AKT2 probe sets are applied to the sample but are not concurrently assayed sequential signals to be assigned code words. Rather, decoding oligos that target the BCAM probe sent are alternately applied with those that target AKT2, such that the code words for BCAM and AKT2 are alternately collected, one at a time, in series. At a particular location, the code word 1001001 is collected using the first decoding oligo treatment, while 0100100 is collected using the second decoding oligo treatment. The code words are assigned to BCAM and to AKT2, respectively, and an in silico overlay of the two decoding oligo set reads indicates that BCAM and AKT2 reside in close proximity to one another within a diffraction limit precluding their distinct detection in a single detection round.

### 7. Example—BCAM-AKT2 Fusion

The BCAM-AKT2 fusion is implicated in cancer progression. In a first run, a tissue is assayed for BCAM and AKT2 transcript localization. Code words are assigned to each target analyte, with 1 representing signal and 0 representing non-signal. No target analyte is assigned a code word having greater than 60% signal. BCAM is assigned a code word of 1001001, while AKT2 is assigned a code word of 0100100. A sample tissue is assayed for BCAM and AKT2 localization. The sample harbors a BCAM-AKT2 fusion, resulting in BCAM probe sets and AKT2 probe sets binding to targets that are adjacent within a diffraction limit precluding their distinct detection in a single detection round.

The code word 1101101 is observed at multiple loci. The code word comprises 5/7 signal letters and 2/7 non-signal letters. This proportion is above a 60% threshold, and the word is identified as indicative of probe sets binding to targets that are adjacent within a diffraction limit precluding their distinct detection in a single detection round. However, the identity of the target at that location is not determined.

In a second run, a tissue is assayed for BCAM and AKT2 transcript localization. BCAM probe sets and AKT2 probe sets are applied to the sample but are not concurrently assayed sequential signals to be assigned code words. Rather, decoding oligos that target the BCAM probe sent are alternately applied with those that target AKT2, such that the code words for BCAM and AKT2 are alternately collected, one at a time, in series. At a particular location, the code word 1001001 is collected using the first decoding oligo treatment, while 0100100 is collected using the second decoding oligo treatment. The code words are assigned to BCAM and to AKT2, respectively, and an in silico overlay of the two decoding oligo set reads indicates that BCAM and AKT2 reside in close proximity to one another within a diffraction limit precluding their distinct detection in a single detection round.

1. A multiplex-method for detecting different analytes in a sample beyond the diffraction limit by sequential signal-encoding of said analytes.

2. The method according to claim 1, wherein different analytes are contacted with at least one set of analyte-specific probes and wherein at least one set of decoding oligonucleotides per analyte per set of multi-decoding oligonucleotides is used.

3. The method according to any one of the previous claims, wherein at least one set of signal oligonucleotides per one set of decoding oligonucleotides per analyte is used.

4. The method according to any one of the previous claims, wherein at least a first set of decoding oligonucleotides and a second set of decoding oligonucleotides is used to identify an analyte in a sample.

5. The method according to any one of the previous claims, wherein at least the first set of decoding oligonucleotides and the second set of decoding oligonucleotides are added to the analyte in consecutive steps.

6. The method according to any one of the previous claims, wherein the signals are optically distinct from each other to allow the detection of different analytes in a sample beyond the diffraction limit.

7. The method according to any one of the previous claims, wherein optical filters and/or computational methods are used in order to differentiate the at least two signals and to allow the detection of different analytes in a sample beyond the diffraction limit.

8. The method according to any one of the previous claims, wherein the detection limit is either because of low spatial distance between each analyte and/or low abundance of at least one of the analytes.

9. The method according to any one of the previous claims, wherein the at least two signals enable the detection of analytes within a sample, which are beyond the detection limit of a single signal.

10. The method according to any one of the previous claims, for detecting different analytes in a sample beyond the diffraction limit by sequential signal-encoding of said analytes, comprising the steps of:
(A1) contacting the sample with a first set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(A2) contacting the sample with a second set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
wherein (optionally) the number of probes and/or targets of first set of analyte-specific probes according to step A1 (the transcript plexity of A1) is at least 10 times higher than the number of probes and/or targets of the second set of analyte-specific probes according to step A2 (the transcript plexity of A2); and
(B1) contacting the sample with at least a first set of decoding oligonucleotides per analyte, wherein in each set of decoding oligonucleotides for an individual analyte each decoding oligonucleotide of the for the first set of analyte-specific probes according to step A1 comprises:
(aa) an identifier connector element (t) comprising a nucleotide sequence which is essentially complementary to at least a section of the unique identifier sequence of the identifier element (T) of the corresponding analyte-specific probe set A1, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(B2) contacting the sample with at least a second set of decoding oligonucleotides per analyte, wherein in each set of decoding oligonucleotides for an individual analyte of each decoding oligonucleotide for the second set of analyte-specific probes according to step A2 comprises:
(aa) an identifier connector element (t) comprising a nucleotide sequence which is essentially complementary to at least a section of the unique identifier sequence of the identifier element (T) of the corresponding analyte-specific probe set A2, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t);
(C) contacting the sample with at least a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element; and
(D) Detecting the signal caused by the signal element;
(E) selectively removing the decoding oligonucleotides and signal oligonucleotides from the sample, thereby essentially maintaining the specific binding of the analyte-specific probes to the analytes to be encoded;
(F) Performing at least three (3) further cycles comprising steps B) to E) to generate an encoding scheme with a code word per analyte, wherein in particular the last cycle may stop with step (D).

11. The method according to any one of the previous claims, wherein steps A1 and A2 as well as steps B1 and B2 can be performed in consecutive cycles of the steps in the order (A1, B1, C, D, E and F) n and then (A2, B2, C, D, E and F) n; or in interwoven cycles of the steps in the order (A1, A2, B1, B2, C, D, E and F) n, wherein n is the number of cycles and at least 3.

12. The method according to any one of the previous claims, for the detection of a cancer selected from adenoid cystic carcinoma, mucoepidermoid carcinoma, follicular thyroid carcinoma, breast carcinoma, Ewing sarcoma, small round cell tumors of bone, synovial sarcoma, glioblastoma multiforme, pilocytic astrocytoma, lung cancer, clear cell renal cell carcinoma, bladder cancer, prostate cancer, ovarian cancer and colorectal cancer and/or any combination thereof.

13. The method according to any one of the previous claims, for in vitro diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases comprising the use of the multiplex method according to the present disclosure.

14. The method according to any one of the previous claims, for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment, said method comprising the use of the multiplex method according to the present disclosure.

15. The method according to any one of the previous claims, wherein an error-correction system is integrated in the binding element(S) and/or identifier element (T) and/or identifier connector element (t) and/or translator element (c) and/or translator connector element (C) and/or signal element.

16. The method according to any one of the previous claims, wherein at least 200 different genes can be identified with 8 or less rounds of detection.

17. A kit for multiplex analyte encoding beyond the diffraction limit, comprising:
(A1) at least a first set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(A2) at least a second set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
wherein the number of probes and/or targets of first set of analyte-specific probes according to step A1 (the transcript plexity of A1) is at least 10 times higher than the number of probes and/or targets of the second set of analyte-specific probes according to step A2 (the transcript plexity of A2); and
(B) at least one set of decoding oligonucleotides per analyte set A1 and A2, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(C) a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.

19. An optical multiplexing system suitable for the method according to claims 1-16, comprising at least: at least one reaction vessel for containing the kits or part of the kits according to claim 17; a detection unit comprising a microscope, in particular a fluorescence microscope; a camera; and a liquid handling device.

20. A method for screening, identifying and/or testing a substance and/or drug comprising:
(a) contacting a test sample comprising a sample with a substance and/or drug
(b) detecting different analytes in a sample by sequential signal-encoding of said analytes with a method according to claims 1-16.

21. A kit comprising:
i) a first population of analyte-specific probe sets for indirect sequential signal-encoding of target analytes in a sample, comprising a first probe set comprising probes that label a first target analyte using a first decoding tag and a second probe set comprising probes that label a second target analyte using a second decoding tag, wherein the first target analyte and the second target analyte are expected to be positioned within a sample at a separation greater than a diffraction limit for a signal generated by a signal probe population comprising signal probes separately linked via decoding probes to the first probe set and the second probe set; and
ii) a second population of analyte-specific probe sets for indirect sequential signal-encoding of target analytes in the sample, comprising a third probe set comprising probes that label a third target analyte, wherein the third target analyte is expected to be positioned within a sample at a separation less than a diffraction limit for a signal generated by a signal probe population comprising signal probes separately linked via decoding probes to the first probe set and the second probe set;
wherein the first population of probes and the second population of probes are not concurrently assayed on a sample.

22. The kit of claim 21, wherein the first population of probes is applied to the sample and subjected to indirect sequential signal-encoding prior to the second population of probes being applied to the sample.

23. The kit of claim 21, wherein the first population of probes is applied to the sample concurrently with the second population of probes, and wherein the first population of probes is not assayed for probe signal concurrently with the second population of probes, such that the first population of probes signal and the second population of probes signal are not simultaneously collected.

24. The kit of claim 23, wherein signal is alternately collected from the first population of probes and the second population of probes.

25. The kit of claim 21, wherein the first population of probes comprises an oncogenic fusion first region targeting probe set, and wherein the second population of probes comprises the oncogenic fusion second region targeting probe set.

26. The kit of claim 21, wherein the first population of probes comprises a transcript first region targeting probe set, and wherein the second population of probes comprises the transcript second region targeting probe set.

27. The kit of claim 26, wherein the transcript second region is alternatively spliced relative to the transcript first region.

28. A method, comprising: contacting a sample to a first population of analyte-specific probe sets for indirect sequential signal-encoding of target analytes in a sample, comprising a first probe set comprising probes that label a first target analyte using a first decoding tag and a second probe set comprising probes that label a second target analyte using a second decoding tag, wherein the first target analyte and the second target analyte are expected to be positioned within a sample at a separation greater than a diffraction limit for a signal generated by a signal probe population comprising signal probes separately linked via decoding probes to the first probe set and the second probe set; and
contacting the sample to a second population of analyte-specific probe sets for indirect sequential signal-encoding of target analytes in the sample, comprising a third probe set comprising probes that label a third target analyte, wherein the third target analyte is expected to be positioned within a sample at a separation less than a diffraction limit for a signal generated by a signal probe population comprising signal probes separately linked via decoding probes to the first probe set and the second probe set;
wherein the first population of probes and the second population of probes are not concurrently assayed on a sample.

29. The method of claim 28, comprising capturing a first iterative label corresponding to the first probe set, and capturing a second iterative label corresponding to the second probe set, wherein no first iterative label component is simultaneously captured with a second iterative label.

30. The method of claim 29, wherein the first iterative label is captured without interruption by capture of a second iterative label component.

31. The method of claim 29, wherein capture of the first iterative label is interrupted by capture of a second iterative label component.

32. The method of claim 29, wherein first iterative label component capture and second iterative label component capture is effected alternately.

33. The method of any one of claim 29 to claim 32, comprising superimposing a first image generated from capture of the first iterative label and a second image generated from capture of the second iterative label.

34. The method of claim 33, wherein the first iterative label and the second iterative label comprise signals of a common wavelength.

35. The method of claim 33, wherein the first iterative label and the second iterative label indicate that the first target analyte and the second target analyte are positioned in the sample at a separation less than a diffraction limit for the signal.

36. A composition comprising a first population of analyte-specific probe sets for indirect sequential signal-encoding of target analytes in a sample, wherein probes of a first probe set target a first target analyte, and wherein no two analyte-specific probe sets are expected to bind to target analytes that have a separation less than a diffraction limit for a signal generated by a signal probe population comprising signal probes separately linked via decoding probes to the two analyte-specific probe sets.

37. The composition of claim 36, comprising at least 100 analyte-specific probe sets.

38. The composition of claim 36, comprising at least 200 analyte-specific probe sets.

39. The composition of claim 36, comprising at least 500 analyte-specific probe sets.

40. The composition of claim 36, comprising at least 1,000 analyte-specific probe sets.

41. The composition of claim 36, comprising at least 10,000 analyte-specific probe sets

42. The composition of claim 36, comprising a second population of analyte-specific probe sets, wherein at least one probe set of the second population binds a second population target analyte expected to have a separation less than a diffraction limit for a signal generated by a signal probe population.

43. The composition of claim 42, wherein the second population of analyte-specific probe sets comprises no more than 1/10 as many analyte-specific probe sets as the first population of analyte-specific probe sets.

44. The composition of claim 42, wherein the second population of analyte-specific probe sets comprises no more than ⅕ as many analyte-specific probe sets as the first population of analyte-specific probe sets.

45. A method of sequential labeling quality control, comprising employing a probe set for sequential labeling of target analytes in a sample wherein probes of the probe set exhibit a minimum proportion of non-signal letters to code words for analytes identified by a probe set for sequential labeling of target analytes in a sample, applying the probe set to a sample to assign code words to target analytes in the sample, determining the code words for the target analytes in the sample, and discarding any code word data comprising code words that exhibit less than the minimum proportion of non-signal letters.

46. A method of sequential labeling quality control, comprising assigning a sequential labeling first code word to a first analyte probe set that identifies the first probe set, and assigning a sequential labeling second code word to a second analyte probe set that identifies the second probe set, such that overlapping simultaneous collection of the first code word and the second code word at a single position beyond the diffraction limit of detection of a signal probe used to generate the first code word and the second code word yields a third code word that identifies the first probe set and the second probe set as being colocalized.

47. The method of claim 46, wherein the first code word is unique.

48. The method of claim 46 or claim 47, wherein the second code word is unique.

49. The method of any one of claims 46-48, wherein the third code word is unique.

50. The method of claim 46, wherein the first code word and the second code word identify a first oncogenic gene fusion partner and a second oncogenic gene fusion partner, respectively.

51. The method of claim 46, wherein the third code word is unique to a oncogenic gene fusion.

52. The method of claim 46, wherein the third code word is unique to a transcript selected from the list consisting of BRC-ABL, TEL-AML, AML1-ETO, BCAM-AKT2, and TMPRSS2-ERG [HERE more examples].

53. A method of increasing sequential labeling reaction resolution, comprising applying a population of target analyte probe sets to a sample, wherein each target analyte probe set has a corresponding unique decoding oligo selected from a decoding oligo population; administering an aliquot of a first subset of the decoding oligo population to the sample, wherein the first subset of the decoding oligo population excludes decoding oligos for at least one target analyte probe set; performing a signal detection on the sample and removing the first subset of the decoding oligo population from the sample; administering an aliquot of a second subset of the decoding oligo population to the sample, wherein the second subset of the decoding oligo population excludes decoding oligos for at least a second target analyte probe set; performing a signal detection on the sample and removing the second subset of the decoding oligo population from the sample.

54. The method of claim 53, comprising repeating steps of administering and performing signal detection such that code words for target analyte probe sets are obtained, and such that letters of code words relying upon the first subset of the decoding oligo population are not simultaneously obtained with letters of code words relying on the second subset of the decoding oligo population.

55. The method of claim 54, wherein letters of code words relying upon the first subset of the decoding oligo population are obtained prior to letters of code words relying upon the second subset of the decoding population.

56. The method of claim 54, wherein letters of code words relying upon the first subset of the decoding oligo population are obtained alternately to letters of code words relying upon the second subset of the decoding population.

57. The method of claim 54, comprising superimposing an image generated from the first code words and an image generated from the second code words.

58. The method of claim 57, wherein the superimposing is effected in silico.

59. The method of claim 54, wherein at least one target analyte identified by a code word relying upon the first subset of the decoding oligo population colocalizes with one target analyte identified by a code word relying upon the second subset of the decoding oligo population.

60. The method of claim 53, wherein the decoding oligo population consists of a first subset and a second subset.

61. The method of claim 53, wherein the decoding oligo population comprises a first subset, a second subset and a third subset.

62. The method of claim 53, wherein the decoding oligo population first subset is selected to identify target analyte probe sets that bind target analytes not expected to colocalize.

63. The method of claim 53, wherein the decoding oligo population first subset is selected to identify at least 5× as many target analyte probe sets as the decoding oligo population second subset.

64. The method of claim 53, wherein the decoding oligo population first subset is selected to identify at least 10× as many target analyte probe sets as the decoding oligo population second subset.

65. A dataset comprising localization information for a plurality of analytes in a sample, wherein the localization information is collected through electromagnetic emission signals collected from the sample, and wherein at least some of the analytes are localized within 10 um of one another in the dataset.

66. The dataset of claim 65, wherein the plurality of analytes comprises at least 1,000 analytes.

67. The dataset of claim 66, wherein the plurality of analytes comprises at least 5,000 analytes.

68. The dataset of claim 67, wherein the plurality of analytes comprises at least 10,000 analytes.

69. The dataset of any one of claims 65-68, wherein at least some of the analytes are localized within 5 um of one another.

70. The dataset of claim 69, wherein at least some of the analytes are localized within 2 um of one another.

71. The dataset of claim 70, wherein at least some of the analytes are localized within 1 um of one another.

72. The dataset of any one of claims 65-71, wherein at least some of the analytes are positioned at a distance that is less than the resolution limit of the electromagnetic emission signals collected from the sample.

73. The dataset of any one of claims 65-72, wherein the dataset comprises data for an image of the sample.

74. The dataset of any one of claims 65-73, wherein the dataset comprises sequence information for at least some of the analytes.

75. The dataset of any one of claims 65-74, wherein the dataset is depicted as an image on a computer monitor or other monitor.

76. The dataset of any one of claims 65-74, wherein the dataset is depicted as an image on a tangible medium.

77. The dataset of any one of claims 65-74, wherein the dataset is depicted holographically.

78. The dataset of any one of claims 65-77, wherein the dataset is generated using fewer wavelengths than are analytes to be analyzed

79. The dataset of claim 78, wherein the dataset is assayed by capturing emission spectra of no more than 10 wavelengths.

80. The dataset of claim 79, wherein the dataset is assayed by capturing emission spectra of no more than 4 wavelengths.

81. The dataset of claim 80, wherein the dataset is assayed by capturing emission spectra of no more than 2 wavelengths.

82. The dataset of any one of claims 65-81, wherein the dataset is generated using a wavelength capture device having no more than 4 channels for wavelength detection.

83. The dataset of any one of claims 65-81, wherein the dataset is generated using a wavelength capture device having no more than 2 channels for wavelength detection.

84. The dataset of any one of claims 65-83, wherein an analyte of the dataset is detected through collection of a series by a series of emission signals that temporally form a ‘word’ indicative or, specifying, or unique to the analyte.

85. The dataset of any one of claims 65-84, wherein the dataset is generated using analyte position information that is collected in at least two temporally distinct collection events.

86. The dataset of any one of claims 65-85, wherein colocalized analytes are indicated.

87. The dataset of claim 86, wherein colocalized analytes are indicated in an image of the dataset.

88. The dataset of claim 86, wherein colocalized analytes are indicated distinctly from an image of the dataset.

89. The dataset of claim 86, wherein colocalized analytes indicate a gene fusion event.

90. The dataset of claim 86, wherein colocalized analytes indicate a chimeric transcript.

91. The dataset of claim 86, wherein colocalized analytes indicate a disease candidate.

92. The dataset of claim 86, wherein colocalized analytes indicate a drug target.
